SEQUENCE LISTING (6 sequences)

SEQ ID NO: 1 - DNA, 20 bp, Artificial Sequence
  Synthetic constructs
tgcccagaaaatgaaaaagg

SEQ ID NO: 2 - DNA, 20 bp, Artificial Sequence
  Synthetic constructs
gtgtatgtggcaatgcgttc

SEQ ID NO: 3 - DNA, 20 bp, Artificial Sequence
  Synthetic constructs
catctgcgagtgtggtgtct

SEQ ID NO: 4 - DNA, 20 bp, Artificial Sequence
  Synthetic construct
ggggtaatttgtcccgactt

SEQ ID NO: 5 - DNA, 26 bp, Artificial Sequence
  Synthetic constructs
catggagtcctggcatccacgaaact

SEQ ID NO: 6 - DNA, 26 bp, Artificial Sequence
  Synthetic constructs
atctccttctgcatcctgtcggcata

(19) United States
(12) Patent Application Publication
(10) Pub. No.: US 2021/0189327 A1
(43) Pub. Date: Jun. 24, 2021

(54) 3D STIMULATED TISSUE CONSTRUCTS AND METHODS OF MAKING THEREOF

(71) Applicant: McMaster University, Hamilton (CA)

(72) Inventors: Ponnambalam Selvaganapathy, Dundas (CA); Alireza Shahin-Shamsabadi, Hamilton (CA)

(21) Appl. No.: 17/132,784

(22) Filed: Dec. 23, 2020

Related U.S. Application Data
(60) Provisional application No. 62/953,245, filed on Dec. 24, 2019.

(51) Int. Cl. C12N 5/00

(52) U.S. Cl. CPC C12N 5/0062; C12N 2537/10; C12N 2533/54; C12N 2513/00

(57) ABSTRACT

This application discloses a method for preparing a construct comprising preparing a mixture of an extracellular matrix and a plurality of cells suspended in a first cell culture medium, applying a crosslinking or gelation agent to the mixture, depositing the mixture into a mold of a defined shape, allowing the extracellular matrix in the mixture to crosslink or gel for a duration of about 1 hour to about 4 hours, applying a second cell culture medium to the mixture containing crosslinked or gelled extracellular matrix, and allowing cell directed self-assembly of the mixture for a duration of about 2 hours to about 10 hours to form a construct, wherein the construct is a three-dimensional structure formed within the mold of the defined shape. Optionally, the method further comprises applying at least one stimuli to the mixture or the construct. Also provided are constructs prepared according to the methods disclosed herein.

### CROSS-REFERENCE TO RELATED APPLICATIONS

The present application claims the benefit of priority from co-pending U.S. provisional patent application No. 62/953,245 filed on Dec. 24, 2019, the contents of which are incorporated herein by reference in their entirety.

### INCORPORATION OF SEQUENCE LISTING

A computer readable form of the Sequence Listing “3244-P60695US01_SequenceListing.txt” (4,096 bytes), submitted via EFS-WEB and created on Dec. 18, 2020, is herein incorporated by reference.

### FIELD

The present application relates to the field of tissue engineering, and in particular, to methods of making and stimulating three-dimensional tissue constructs and uses thereof.

### BACKGROUND

Although monolayer or two-dimensional (2D) cell culture models are considered to be the gold standard for in vitro modeling of pathophysiological events, they cannot reconstruct in vivo like gradient of gases and nutrients and lack proper cell-cell and cell-matrix interactions. Three-dimensional (3D) cell culture techniques were developed to better model biological behavior. For instance, only 3D systems show drug responses and gene expression patterns that are comparable to in vivo systems. This is because the tissue topography and cell-cell interactions in 3D systems are more biomimetic compared to 2D ones.

Spherical cellular aggregates, otherwise known as multicellular spheroids, are one of these 3D in vitro models that are formed when cells are cultured in suspension or non-adherent surfaces. These spheroids are cell aggregates containing complex cell-cell and cell-matrix interactions that recapitulate natural microenvironments of cells including natural gradient of nutrients, gases, and different growth and signaling factors that are physiologically relevant. Microstructure of spheroids can be manipulated to study the effect of chemical, physical, physiological, and architectural environment on different cellular function and behavior. Thus, multicellular spheroids, are widely used as three-dimensional in vitro models to mimic natural in vivo cellular microenvironment for applications such as drug screening.

Different techniques for multicellular spheroid formation can be classified based on whether they incorporate extracellular matrices (ECM) in their initial construction. Matrix-free methods, in which a dispersion of cells initially form loose aggregates and slowly turn into more solid structures over a period of days due to establishing cell-cell interactions and subsequent generation and assembly of their own extracellular matrices, are more common. These include hanging drops, spinner flasks, low adhesion flasks, and external force-driven techniques. However, matrix-free techniques sometimes form spheroids with uncontrolled morphologies and low structural reproducibility. Moreover, aggregation is also limited in the size that they can grow, the types of cells, and the matrix composition secreted by the cells. Therefore, these systems are more suited for developmental studies and for use with stem cells.

Alternatively, matrix-based techniques start with cells embedded in a hydrogel matrix such as collagen, Matrigel™, or alginate that serves as the scaffold and provides the shape of the construct formed. Such techniques have the advantage of high control over cell and ECM source and type, size and shape of the formed structures, cell density and are more suited to tailoring the cellular microenvironment to simulate in vivo conditions for studying disease processes and drug discovery. An emerging matrix-based technique of forming spheroids is using micro-fabricated molds which can reduce the amount of reagents used with a high cell to ECM solution ratio that decreases the amount of shear stress applied to the cells, while allowing scale up and standardization of spheroid generation. However, such techniques are time consuming (spheroid formation usually takes a few days), limited in cell type and low cell density, and show no or limited control over positioning different type of cells in the 3D structure. Furthermore, the spherical shape of most of these constructs ensure that they can only be grown up to a certain size beyond which a necrotic core forms due to transport limitations.

A simple and scalable technique capable of forming tissue constructs of various 3D shapes from a wide variety of cell types at physiologically-relevant cell densities and architecture through precise control of the cell distribution and spatial arrangement, including those of different cell types, would be useful to overcome these limitations and provide a more relevant 3D model.

### SUMMARY

In the present application, matrix- and cell-directed self-assembly processes are combined to develop a rapid, scalable, controllable, and simple method to form multicellular tissue constructs. The self-assembly process is rapid and is typically completed within a few hours, as opposed to days for other methods, which produces a mechanically robust tissue construct that could be handled easily. The method is capable of forming constructs in a variety of shapes such as spheres, rods, dumbbells and cuboids, and can be easily parallelized to produce large numbers at the same time. It also has the flexibility to produce both homogeneous multicellular constructs as well as heterogeneous ones wherein the location of different types of cells can be precisely defined. These constructs can be made at high and physiologically relevant cell densities with predefined spatial positioning which makes this method appropriate for creating 3D in vitro models for drug discovery applications and biological assays as well as tissue grafts for implantation. The constructs also maintain their shape even after being removed from the mold in which they were formed.

Accordingly, the present application discloses a method for preparing a construct comprising a) preparing a mixture of an extracellular matrix and a plurality of cells suspended in a first cell culture medium, b) applying a crosslinking or gelation agent to the mixture, c) depositing the mixture from b) into a mold of a defined shape, d) allowing the extracellular matrix in the mixture in c) to crosslink or gel for a duration of about 1 hour to about 4 hours, e) applying an additional cell culture medium to the mixture from d) containing crosslinked or gelled extracellular matrix, and f) allowing cell directed self-assembly of the mixture from e) for a duration of about 2 hours to about 10 hours to form a construct, wherein the construct is a three-dimensional structure formed within the mold of the defined shape.

In one embodiment, the method further comprises removing the construct from the mold.

In one another embodiment, the construct retains the defined shape after removal from the mold.

In another embodiment, the extracellular matrix comprises a hydrogel, collagen, fibrin, laminin, elastin, alginate, gelatin, fibrinogen, chitosan, hyaluronan acid, polyethylene glycol, lactic acid, N-isopropyl acrylamide, glycoproteins, proteoglycans, basement membrane proteins, Matrigel™, Geltrex™, or combinations thereof. In another embodiment, the ratio of the volume of the volume of the extracellular matrix to the volume of the first cell culture medium is between 1:1 and 1:10, optionally 1:1 to 1:6. The extracellular matrix may have a concentration of about 4 mg/mL to about 20 mg/mL.

In another embodiment, the gelation agent is an alkaline substance, optionally NaOH, which increases the pH of the mixture to 7.2-7.4.

In another embodiment, 10⁵ to 10¹⁰ cells/mL are suspended in the first cell culture medium.

In another embodiment, the plurality of cells comprise mammalian cells, optionally hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, immune cells, vascular cells, pluripotent cells and stem cells, cancer cells and tumor cells, or combinations thereof.

In another embodiment, the construct comprises about 10⁶ cells/mL to about 10¹⁰ cells/mL.

In another embodiment, the plurality of cells are selectively positioned within the construct in a defined manner.

In another embodiment, the plurality of cells comprise the same cell type.

In another embodiment, the construct comprises different cell types existing as a homogenous mixture within the construct.

In another embodiment, the construct comprises different cell types spatially separated within the construct.

In a further embodiment, the method further comprises preparing at least one additional mixture of a second extracellular matrix and a second plurality of cells suspended in a second cell culture medium, wherein the second plurality of cells comprise at least one different cell type from the plurality of cells, applying a crosslinking or gelation agent to the additional mixture and depositing the additional mixture into the mold such that different cell types are spatially separated within the construct.

In another embodiment, the cell culture medium comprises a basal medium and optionally at least one supplement selected from plasma, serum, lymph, amniotic fluid, pleural fluid, growth factors, hormones, crude protein fractions, recombinant proteins, protein hydrolysates, synthetic polypeptide mixtures, tissue extracts and combinations thereof. Optionally, the first cell culture medium and the additional cell culture medium are the same.

In another embodiment, the cell culture medium comprises natural biological substances selected from plasma, serum, lymph, amniotic fluid, pleural fluid, growth factors, hormones, crude protein fractions, recombinant proteins, protein hydrolysates, tissue extracts or combinations thereof.

In another embodiment, the mold or parts of the mold comprise a material that is removed from the construct after the three-dimensional structure is formed, optionally wherein the material is extracted, dissolved or melted from the three-dimensional structure of the construct.

In another embodiment, the mold comprises a cell non-adhesive material, optionally polydimethylsiloxane.

In another embodiment, the mold defines the shape of a sphere, rod, tube, dumbbell, cuboid or combination thereof.

In another embodiment, the mold comprises a wire or rod that when removed from the construct after the three-dimensional structure is formed, results in a construct comprising a hollow interior space.

In another embodiment, the mold is prepared using microfabrication.

In another embodiment, at least one stimuli is applied to the mixture or the construct, optionally wherein the stimuli is a biophysical stimuli.

In one embodiment, the mold defines the shape of a tube and at least one elongated metal material is inserted into the mold.

In another embodiment, at least one stimuli is applied to the mixture or the construct via the at least one elongated metal material.

In another embodiment, the construct is used in vitro for research and development.

In another embodiment, the construct is used in vivo for cell therapy.

Also provided in the present application are constructs prepared according to the methods disclosed herein.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows schematics of a) the fabrication process involving addition of collagen, DMEM as culture medium, and cell mixture to wells; b) the expected shrinkage pattern and additional steps of the process with time; c) mono- and co-culture of cells formed as 3D constructs/spheroids; d) different construct morphologies formed with this technique; e) constructs made with precise control over distribution of cells in complex structures in exemplary embodiments of the application.

FIG. 2 shows shrinkage of grafts with time for different well sizes and cell numbers in exemplary embodiments of the application.

FIG. 3 shows a) bright field images of spheroids at beginning and end of the process; b) final radius of spheroids normalized to initial radius (n=6; P-values: *<0.01, + is 0.01); c) effect of well size (2.5 and 4 mm in diameter) and cell number (10⁵ and 10⁶) on shrinkage pattern (normalized radius with time) in exemplary embodiments of the application.

FIG. 4 shows the shrinkage of collagen in absence of cells (methylene blue was added for observation purposes) in exemplary embodiments of the application.

FIG. 5 shows the shrinkage pattern of 5×10⁴ cells in small and large wells as the lower limit in exemplary embodiments of the application.

FIG. 6 shows the effect of collagen to medium ratio on the final spheroid size (1:1 and 1:3 ratio of collagen to medium for 5×10⁴ cells in large wells) in exemplary embodiments of the application.

FIG. 7 shows a) live stained spheroids at the end of the fabrication process (6 hrs after process is started); b) H&E stained sections of different MCF-7 spheroids showing compactness of cells in exemplary embodiments of the application.

FIG. 8 shows a) total protein content spheroids with and without MCF-7 cells; b) total metabolic activity of the spheroids (all of P-values are <0.01) in exemplary embodiments of the application.

FIG. 9 shows a) expression of Cadherins and Integrins using PCR (n=4; P-values: *<0.01); b) spheroids of cells with disrupted actin networks (n=6 with no significant difference between different conditions) in exemplary embodiments of the application.

FIG. 10 shows a) effect of cell type on final spheroid radius in large wells with 10⁵ Cells after 6 hrs (n=6; P-values: * is 0.01, + is 0.08, \hat{ } is 0.02, and is 0.04); b) final spheroids of HUVEC, HS578T, SaOS-2, and MDA cell lines that formed spheroids; c) grafts of cells that did not form spheroids at 6 hrs (L929, C2C12, 3T3, and CHO cells) in exemplary embodiments of the application.

FIG. 11 shows a) setup for mechanical testing; b) comparison of spheroids in terms of their stiffness (n=8) in exemplary embodiments of the application.

FIG. 12 shows a) brightfield and fluorescent images of 3T3 and HUVEC (10% of total population) cells co-culture with MCF-7 cells in large wells with 10⁵ cells after 6 hrs; b) Effect of second cell type on final spheroid radius (n=6; P-values: *<0.01) in exemplary embodiments of the application.

FIG. 13 shows collagen versus Geltrex™ and dispersion in DMEM in fabrication of the spheroids in exemplary embodiments of the application.

FIG. 14 shows different morphologies formed with DMEM to collagen ratio of 1:1 in exemplary embodiments of the application: a) dumbbell with 10⁶ cells in 60 μL bioink, b) cross with 10⁶ cells in 50 μL bioink, c) cuboids with 5×10⁵ and 10⁶ cells in 8 and 16 μL bioink.

FIG. 15 shows a graft in the shape a cross out of PDMS wells after a) 24 hrs, b) 3 days, c) 7 days demonstrating that without constraints of the well the constructs maintained the predefined shape in exemplary embodiments of the application.

FIG. 16 shows a) connected wells with gfp-3T3 cells in left, MCF-7 cells stained with blue tracker in center, and rfp-HUVEC cells; b) dumbbell formation with time and defined borders between cell types in exemplary embodiments of the application.

FIG. 17 shows a schematic of the process for forming tubular constructs in exemplary embodiments of the application: a) silicon tubing is filled with neutralized collagen, medium, and cell solution; b) after collagen gels and cells adhere to it, collagenous construct is formed within the tubing by clinging to stainless-steel pins inserted in the tubing as support; c) fluid flow, electrical stimuli, and deformation of tubing (stretching, bending, and torsion) can be applied to create a 3D dynamic environment for cells.

FIG. 18 shows characterization of parameters effective on the “tissue-in-a-tube” process described herein using MCF-7 cells in exemplary embodiments of the application: effect of a) cell density (with 1:3 collagen to medium ratio (CMR) and medium thickness tubing); b) CMR (with density of 2×10⁶ cells/mL and medium thickness tubing); c) tubing thickness (with 1:3 CMR and density of 2×10⁶ cells/mL); d) effect of distance between anchor pins, longer constructs can be formed by increasing the length of the tubing (with 1:3 CMR and density of 1×10⁶ cells/mL); e) Live/dead stained samples 4 hrs after process was started; f) increasing the cell density would increase the contraction which leads into developing a tear in the structure—all images are taken 4 hrs after assembly; in each case n=4 was used to ensure the process is repeatable.

FIG. 19 shows shrinkage pattern of the constructs over time for samples formed with MCF-7 cells with 2×10⁶ cells/mL and 1:3 ratio in exemplary embodiments of the application.

FIG. 20 shows distribution of live and dead cells in the middle of the construct vs. close to anchor points (staining and imaging were done 4 hrs after the fabrication process started) in exemplary embodiments of the application.

FIG. 21 shows a) controlled graft interfaces containing different cells in tissue-in-a-tube constructs in Axial and Radial configurations with clear continuity and interfaces; b) failed and robust interfaces in constructs with Axial configuration; c) anchor point formed with two cells in Radial configuration in exemplary embodiments of the application (all the images in panels b and c are taken after 8 hrs of starting the process).

FIG. 22 shows formation of macrostructures with complex patterns using tissue-in-a-tube technique in exemplary embodiments of the application: a) long column with descending thickness and b) with bifurcation; constructs are formed with HUVECs (with density of 2×10⁶ cells/mL and 1:3 CMR) and are stable outside the tubing.

FIG. 23 shows a) components of the bioreactor: Arduino microcontroller creates the AC step signal (50 Hz and −5 to +5 V) and controls the flow rate through the speed of the motor that can be defined using the potentiometer and is shown on the LCD; b) assembled bioreactor in exemplary embodiments of the application.

FIG. 24 shows the effect of dynamic microenvironment on cellular constructs in exemplary embodiments of the application: a) constructs were formed with undifferentiated myoblast C2C12s and differentiation was performed in three different conditions: “In Well” with no constrictions, “In Tube”, constricted between anchor points, and in “Dynamic” condition anchored to the pins and facing electrical stimuli; b) effect of culture condition on thickness of the constructs, *P-value<0.01 (n=4); c) total protein content of differentiated C2C12s in three culture conditions, **P-value<0.001 (n=4); d) effect of culture condition on formation of multinucleated muscle fibers and their alignment; e) Live/dead stained images of “In Tube” and “Dynamic” samples at day 4 right after retrieving from the tubing (slightly more dead cells are observed in the “In Tube” group); f) effect of electric field on alignment of SH-SY5Y and Saos-2 cells.

FIG. 25 shows a) a schematic of the mechanical deformation imposed to C2C12 grafts to create a dynamic microenvironment and b) effect of mechanical deformation on fiber formation of skeletal muscle grafts in dynamic environment as compared to static condition in exemplary embodiments of the application—stimulation was started one day after grafts were formed and a dynamic environment was created by deforming the tubing with amplitude of 2 cm and frequency of 0.5 Hz for 2 hr every day for three days.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

### II. Methods and Compositions of the Application

In the present application, a rapid fabrication method has been developed to form spatially-controlled multicellular tissue constructs through self-assembly. The process is applicable using different cell types to form complex shapes with predefined distribution of cells and highly controlled interfaces. The self-assembly of extracellular matrix, such as collagen, that forms the scaffold attaching cells and addition of a follow-up dose of growth medium were found to be important for this rapid fabrication method. Spherical and non-spherical constructs, which are robust and retain their shape even after removal from the mold, can be formed. Both homogeneous and heterogeneous multicellular constructs can be constructed, which is useful as a realistic in vitro model for bioassays that investigate the interaction between different cell types. The heterogeneous constructs not only provide precise spatial positioning but also sharp interfaces which can be important in quantification of migration, or gene and protein expression in these bioassays. Such heterogeneous constructs provide physiologically relevant cell densities, 3D structure as well as close positioning of multiple types of cells that are not possible using other fabrication approaches. Low to very high cell numbers can be used in small or larger structures to appropriately tune cell density to be physiologically relevant for different applications such as tissue development or drug screening. Although these constructs can be immediately applied as 3D in vitro models for drug discovery, the method can also be adapted for use in regenerative medicine, for example, as tissue grafts for implantation.

In one aspect of the application, provided is a method for preparing a construct (for example, a cell or tissue construct) comprising preparing a mixture of an extracellular matrix and a plurality of cells suspended in a first cell culture medium, applying a crosslinking or gelation agent to the mixture, depositing the mixture into a mold of a defined shape, allowing the extracellular matrix in the mixture to crosslink or gel for a duration of about 1 hour to about 4 hours, applying an additional cell culture medium to the mixture containing crosslinked or gelled extracellular matrix, allowing cell directed self-assembly of the mixture for a duration of about 2 hours to about 10 hours to form a construct, wherein the construct is a three-dimensional structure formed within the mold of the defined shape.

As used herein, the term “extracellular matrix” or “ECM” refers to a non-cellular support material. In one embodiment, the extracellular matrix comprises a hydrogel. In another embodiment, the extracellular matrix gel comprises collagen, fibrin, laminin, elastin, alginate, gelatin, fibrinogen, chitosan, hyaluronan acid, polyethylene glycol, lactic acid, N-isopropyl acrylamide, glycoproteins, proteoglycans, basement membrane proteins, Matrigel™, Geltrex™, or combinations thereof. In some embodiments, the extracellular matrix has a concentration of about 4 mg/mL to about 20 mg/mL, optionally 5 to 10 mg/mL.

In one embodiment of the method, 10⁵ to about 10¹⁰ cells/mL, optionally 10⁶ to 10⁷ cells/mL, are suspended in the first cell culture medium. Using a high number of cells at the beginning of the process rather than allowing the cells to reach the required cell number shortens the process and can result in a more homogenous cell population. In one embodiment, the final construct has a concentration of 10⁷ to 10¹⁰ cells/mL.

In one embodiment, the plurality of cells comprise mammalian cells. In another embodiment, the plurality of cells include cells selected from the group consisting of hepatocytes, pancreatic Islet cells, fibroblasts, chondrocytes, osteoblasts, endothelial cells, exocrine cells, smooth or skeletal muscle cells, myocytes, adipocytes, ectodermal cells, ductile cells, kidney cells, intestinal cells, parathyroid and thyroid cells, nerve cells, ocular cells, integumentary cells, immune cells, vascular cells, pluripotent cells and stem cells, cancer cells and tumor cells, or combinations thereof.

The plurality of cells may comprise cells of the same cell type, or cells of different cell, tissue and/or organ type. This technique can be used with primary cell lines or differentiated stem cells including induced pluripotent stem cells, embryonic stem cells and adult stem cells, as well as different immortalized cell lines from different tissue types and phenotypes.

In one embodiment, the ratio of the volume of the extracellular matrix to the volume of the first cell culture medium is between 1:1 and 1:10, optionally 1:1 to 1:6 or 1:1 to 1:3. In another embodiment, the ratio of the volume of the volume of the extracellular matrix to the volume of the first cell culture medium is 1:n with n>=1.

The term “bioink” as used herein refers to a mixture comprising cells, extracellular matrix and cell culture medium. A “bioink” may further comprise a crosslinking and/or a gelation agent.

As used herein, the term “cell culture medium” refers to a liquid or semi-solid designed to support the growth of cells. A cell culture medium that is suitable for the specific cell type(s) of the plurality of cells may be used. In one embodiment, the cell culture medium comprises natural biological substances selected from the group consisting of plasma, serum, lymph, amniotic fluid, pleural fluid, growth factors, hormones, crude protein fractions, recombinant proteins, protein hydrolysates, tissue extracts or combinations thereof. In another embodiment, the cell culture medium comprises a basal medium and supplements selected from the group consisting of plasma, serum, lymph, amniotic fluid, pleural fluid, growth factors, hormones, crude protein fractions, recombinant proteins, protein hydrolysates, tissue extracts or a combination thereof. Examples of cell culture media useful in the present methods include, but are not limited to, Dulbecco's Modified Eagle Medium (DMEM), supplemented for example with 10% V/V fetal bovine serum (FBS) and 1% Penicillin-Streptomycin, EBM-2 medium, and McCoy's medium supplemented for example with 15% V/V fetal bovine serum (FBS) and 1% Penicillin-Streptomycin.

In addition to the initial cell-containing cell culture medium (also referred to here as a “first cell culture medium”) which is mixed with the extracellular matrix, a second volume of cell culture medium (also referred to herein as an “additional cell culture medium”) may be added after the extracellular matrix has been crosslinked or gelled. This additional volume of cell culture medium can help to provide the cells with additional nutrients for the remainder of the assembly process.

In one embodiment, the first and the additional cell culture medium are the same. In another embodiment, the additional medium is different from the first medium and may be used, for example, to induce differentiation of cells in the construct.

In one embodiment, a crosslinking or gelation agent is applied to the mixture of the extracellular matrix and the plurality of cells suspended in the first cell culture medium.

The term “gelation agent” as used herein refers to any substance, molecule, atom, or ion that is capable of creating proper environment so that different polymer chains can bind to each other directly or using an external molecule. In one embodiment the gelation agent is an alkaline substance, such as sodium hydroxide (NaOH). In one embodiment, the gelation agent is a solution of sodium hydroxide (0.1-0.5 M) in deionized water. The alkaline substance may be used to adjust the pH of the mixture may be adjusted to at 7.2-7.4, optionally 7.4 or about 7.4. Adjusting the pH to 7.2-7.4 may help to initiate collagen self-assembly.

The term “crosslinking agent” as used herein includes any substance, molecule, atom, or ion that is capable of forming one or more crosslinks between polymer chains. The term “crosslink(s)” or “crosslinking” refers to a comparatively short connecting unit (as in a chemical bond or chemically bonded group), in relation to a monomer, oligomer, or polymer, between neighboring chains of atoms in one or more complex chemical molecule, e.g., a polymers.

Following application of the crosslinking or gelation agent to the mixture, the mixture may be deposited, or filled, into a mold of a defined shape. The mold is not limited to any specific shape or design. It may, for example, define the shape of a sphere, oval, rod, tube, dumbbell, cuboid, cross or variations or combinations thereof. The mold may also define a hollow space, such as a hollow tube. For example, the mold may include a wire or rod shaped material which, when removed from the construct, leaves a hollow space or channel.

The mold is optionally fabricated from a cell non-adhesive material, for example polydimethylsiloxane. In one embodiment, the mold is silicone, for example a silicone tube. The mold may be prepared by any method known in the art, including, for example microfabrication and 3D printing.

The mixture (also referred to herein as a “bioink”) may be deposited, or filled, into the mold by any means known in the art. In one embodiment, a pipette is used to deposit the mixture. In another embodiment, a syringe is used, for example a syringe with a proper gauge needle, such as 18-22. The bioink may be deposited uniformly or in a specific pattern. For example, the bioink may be selectively positioned within the mold in a defined manner. Further, different bioinks comprising different cell types may be selectively positioned in the mold such that they are spatially separated within the resulting construct. This can allow for multiple cell types in a construct in predefined patterns with sub millimeter accuracies and very clear cell-cell interfaces. These interfaces can be preserved for very long times.

Accordingly, in one embodiment, the method comprises preparing at least one additional mixture of a second extracellular matrix and a second plurality of cells suspended in a second cell culture medium, wherein the second plurality of cells comprise at least one different cell type from the plurality of cells, applying a crosslinking or gelation agent to the additional mixture and depositing the additional mixture into the mold such that different cell types are positioned in the mold such that they are spatially separated within the resulting construct. The same extracellular matrix may be used for the original mixture and the additional mixture, or different extracellular matrices may be used. Likewise, the same cell culture medium and/or the same crosslinking or gelation agent may be used for the original mixture and the additional mixture or different cell culture medium and/or crosslinking or gelation agents may be used. The same method may be used to deposit at least 3, 4, 5, or more different cell types in the mold.

Alternatively, different cell types may exist as a homogenous mixture within the construct for example by preparing a bioink comprising different cell types before the bioink is deposited in the mold.

After the mixture is deposited into the mold, it is allowed to crosslink or gel for a duration of about 1 hour to about 4 hours, optionally about 1.5 to about 3 hours or about 2 hours. In one embodiment, the mixture is incubated at 37° C. with 5% CO₂.

In one embodiment, a second volume of cell culture medium is added to the crosslinked or gelled extracellular matrix to provide additional nutrients to the cells. After the additional volume of cell culture medium is added, cell directed self-assembly of the mixture for a duration of about 2 hours to about 12 hours, optionally about 3 hours to about 5 hours or about 4 hours is allowed to occur. In one embodiment, the mixture is incubated at 37° C. with 5% CO₂. During this time, consolidation of the construct occurs to its final state while retaining the 3D shape fixed due to the initial collagen crosslinking or gelation.

The term “consolidation” or “consolidated” as used herein refers to the binding of cells, cell aggregates, multicellular aggregates, multicellular bodies, and/or layers thereof as an integrated structure though to cell-cell and/or cell-ECM attachments. In some embodiments, consolidation involves reduction in volume and/or physical shrinking of the integrated structure.

In some embodiments, the method further comprises removing the construct from the mold, wherein the construct retains a defined shape after removal from the mold. In some embodiments, the mold comprises a material that is removed from the construct after the three-dimensional structure is formed. In some embodiments, the material is extracted, dissolved or melted from the three-dimensional structure of the construct to form hollow constructs.

In some embodiments, the method is capable of forming constructs in a variety of shapes such as spheres, rods, tubes, dumbbells, cuboids or variations or combinations thereof, and can be easily parallelized to produce large numbers at the same time. In some embodiments, the method further comprises forming layered constructs comprising layers made of different compositions (i.e. cell type, extracellular matrix). In some embodiments, the method further comprises forming layered co-axial tubular constructs comprising a plurality of tubular/sheath structures.

In some embodiments, the construct comprises about 10⁶ to about 10¹⁰ cells/mL.

In some embodiments, the method is capable of forming constructs which define a hollow interior space by extracting a wire or rod-shaped mold from the construct to create a hollow space or a channel.

On one embodiment, the method further comprises applying a stimuli to the bioink or the construct. Such a stimuli can be useful to provide a physiological-like cue required to properly recreate the in vivo microenvironment of tissue and organs. The stimuli may for example be a biophysical stimuli such as electrical stimulation. The stimuli may alternatively, or additionally be fluid flow (for example, perfusion of medium, that generate shear force of fluid flow on the cells), or deformation of the mold (for example, mechanical stretching, bending, torsion and/or compression).

In one embodiment, at least one elongated metal material such as a pin (for example a stainless steel pin) or wire, is inserted in to the mold, allowing a stimuli such as mechanical or electrical stimulation to be applied through the elongated metal material. Then, when the bioink gels, the construct may be formed and hang between them and then mechanical or electrical stimulation may be applied through the metallic pin or wire. In one embodiment, two elongated metal materials are inserted into the mold so that when the bioink gels, the construct may be formed and hang between them and then mechanical or electrical stimulation is applied through the elongated metal material. In one embodiment, the stimulation is applied to the mixture after the steps of gelation and consolidation are performed. In another embodiment, the stimulation is applied to the resulting construct.

A stimuli may be applied one or multiple times. A stimuli may also be applied continuously over a period of time. Stimuli may be applied separately or different stimuli (for example, electrical and mechanical stimulation) may be applied at the same time. Electrical and/or mechanical stimulation may also be applied at the same time as perfusion/fluid flow.

In one example, an electrical stimuli with a peak to peak voltage of up to about 10 V and a frequency of up to about 50 Hz is applied to a construct.

After removal from the mold, the construct may maintain its shape for at least one day, two days, 5 days, 7 days, 10 days or two weeks independent of any anchorage.

In one particular embodiment, the mold is tubing such as gas permeable silicone tubing. The tubing optionally has an inner diameter of 0.1 to 10 mm, optionally 1 to 7 mm and/or a length of 0.5 cm to 5 cm, optionally 1 to 3 cm. Elongated metal material such as stainless steel wire 304 “pins” with a 0.5 mm diameter is optionally inserted into the tubing. For example, two pins perpendicular to each other may be inserted into the tubing at two point approximately 1 to 7 cm or optionally 2 to 4 cm apart. A bioink may be deposited in the tubular mold using methods as described herein to form a construct.

Stimuli is optionally applied to the construct during or after its formation. For example, mechanical deformation of the tube such as stretching, bending or torsion may be applied. In another embodiment, the pins are connected to a microcontroller to allow application of electrical stimulation to the construct.

In one embodiment, the method further comprises incubating the construct with an appropriate growth media. In one embodiment, the construct is placed in a container such as a petri dish and immersed in a growth media. In another embodiment, media is added to a channel/hollow space in the construct.

In another aspect of the application, provided are constructs prepared according to the method disclosed herein.

In some embodiments, the construct is used in vitro for research and development, such as for modeling cellular interactions in understanding disease and drug discovery. In some embodiments, the construct is used in vivo for cell therapy, such as tissue grafts and artificial organs for implantation.

The constructs described herein can be used for drug screening. Accordingly, also provided herein is a method for screening for activity of a compound of interest comprising treating a construct as described herein with a compound of interest and observing the effect of the compound on the plurality of cells. For example, a compound of interest may be screened for its effect on the growth rate of the cells, the viability of the cells and/or protein expression in the cells. In one embodiment, different doses of the compound of interest may be studied. The compound of interest is optionally a drug candidate, including for example, a small molecule or a biologics.

The constructs described herein can also be used as in vivo or in vitro bioreactors where cells producing specific biomaterials for example, a protein (for example, an antibody), peptide, hormone (for example, insulin), nucleic acid or lipid are included in the biocompatible gel. Accordingly, in such an embodiment, the methods described herein further comprise culturing the construct and isolating a biomaterial of interest.

The constructs described herein can be further used in regenerative medicine. Accordingly, in such an embodiment, the methods described herein further comprise administering the construct to a subject in need thereof.

Also provided herein are methods of using the constructs of the disclosure as in vitro experimental models.

### EXAMPLES

The following non-limiting examples are illustrative of the present application:

### Example 1. Materials and Methods

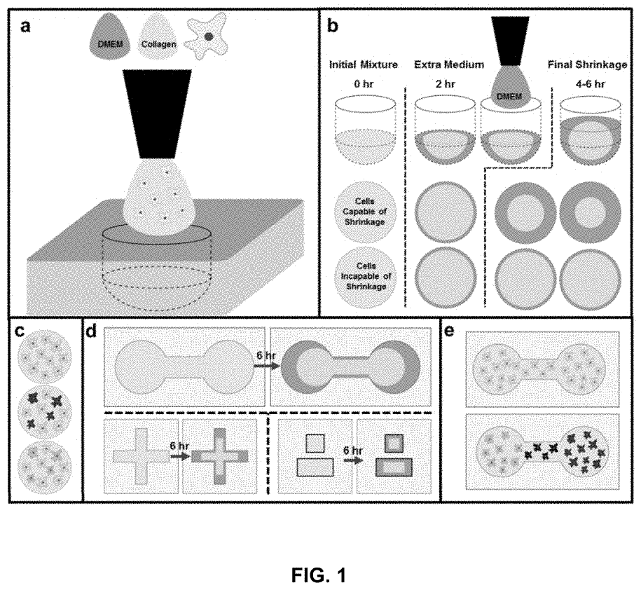

Fabrication process of the 3D Tissue Construct. A two-step fabrication process was devised so that the process of self-assembly associated with the ECM and the cells occurs at different stages. The process begins with the fabrication of molds of appropriate shapes that are representative of the final shapes of the tissue constructs. The molds are made of polydimethylsiloxane (PDMS) that is cast onto 3D printed features that represent the final shape. 3D printing was used for this purpose rather than other replication techniques such as soft-lithography because they are labor intensive and costly and require specific facilities. PDMS provides the low adhesion surface that is important for formation of the tissue construct. Next, the bioink which is composed of a 1:1 mixture of cell loaded (5×10⁴-1×10⁶ cells) culture medium and collagen (5 mg/mL) is filled into the PDMS mold (FIG. 1a). The pH of the bioink was adjusted to 7.4 by adding 0.1 M NaOH immediately before the filling process to initiate collagen self-assembly. Incubation for 2 hrs completes the crosslinking or gelation process and is used to fix the shape of the final tissue construct. Subsequently, extra culture medium was added to provide the cells with sufficient nutrients for the rest of the process. After this addition, a rapid consolidation and shrinkage of the tissue construct happens (within 4 hrs) to its final state while retaining the 3D shape fixed due to the initial collagen crosslinking or gelation (FIG. 1b). The rapid consolidation does not happen in the absence of cells and is related to the cell concentration, indicating that the cell-ECM interaction is primarily responsible for this shrinkage. The bioink can be composed to a single cell type or multiple cell types to create homogeneous mono- and co-culture constructs (FIG. 1c). The shape of the mold fixes the shape of the initial construct formed due to collagen gelation or crosslinking and a variety of 3D shapes including cuboids, dumbbells, and crosses (FIG. 1d) can be formed. Heterogeneous constructs can also be formed by depositing various bioinks composed of different cells in specific locations into the mold (FIG. 1e). The high viscosity of the inks and the hydrophobic nature of the PDMS enable spatial localization of the inks both during the deposition process as well as during the initial collagen crosslinking/gelation and construct formation (FIG. 1e).

Rapid Formation of Spheroidal Tissue Constructs. In order to form spheroidal tissue constructs, PDMS molds in the form of circular wells with spherical bottom were used. These molds were prepared by mixing PDMS and its curing agent with the ratio of 10:1 and casting it on the Poly lactic acid (PLA) master mold with negative of the required patterns. Master molds were designed using software Solidworks© and 3D printed using a Stereolithography (SLA) based 3D printer (Objet 24 Desktop 3D printer). A large and small well size were created with diameters of 4 and 2.5 mm respectively. MCF-7 (Michigan Cancer Foundation-7, a breast cancer cell line) cells were cultured in Dulbecco's Modified Eagle Medium (DMEM) (Thermofisher, high glucose) supplemented with 10% V/V fetal bovine serum (FBS) (Thermofisher, US origin) and 1% Penicillin-Streptomycin (Thermofisher, 10000 U/mL) until 70% confluent. Cells were trypsinized and detached from tissue culture flasks. Required number of cells (10⁵ or 10⁶) were aliquoted, precipitated using centrifugation, and resuspended in proper amount of medium (5 μL for small wells, 15 μL for large wells). The cell solution then was added to the equal amount of bovine collagen type I (Thermofisher, 5 mg/mL) and mixed to get a uniform distribution of cells. Finally, pH of the solution was adjusted to 7.4 by adding sufficient amount of 0.1 M NaOH solution and the mold was incubated at 37° C. with 5% CO₂. Two hrs later, after the collagen was gelled, some DMEM growth medium (25 μL for large wells and 10 μL for small wells) was added to provide the cells with enough nutrients for the remainder of the assembly process. Bright field images of each well were taken using a stereo microscope immediately after filling the wells and after 1, 2, 4, and 6 hrs to measure the shrinkage using ImageJ software. After 6 hrs spheroids were moved to 96 well plates for further applications or observations. The same process was performed with 5×10⁴ cells in small and large wells with 1:1 ratio of collagen to culture medium, as well as 5×10⁴ cells in large wells with 1:3 ratio of the solutions but the same total volume in order to investigate possibility of using this technique with lower cell number or lower ECM to cell ratios.

Viability and Distribution of the Cells in the Final Spheroids. Effect of cell number and well size on cell viability at the end of the process (after 6 hrs) was studied by staining the spheroids (formed in large well with 10⁵ cells and small well with 10⁶ cells) with calcein-AM (Thermofisher). Five μL of calcein-AM solution was dissolved in 5 mL PBS and 100 μL of this solution was added to each spheroid. Spheroids were kept with this solution for 1 hr and then washed with PBS. Images were taken using an upright fluorescent microscope using green fluorescent filter with 4× magnification.

Microstructure of the spheroids was compared by studying two set of spheroids created with different cell densities. The high-density spheroid was fabricated in small wells loaded with 10⁶ cells, while the low-density spheroids were fabricated in large wells with 10′ cells. For histological staining, at the end of 6 hr process, spheroids were fixed in 4% wt/V formaldehyde in DI water for 1 hr, dehydrated step wise in 40, 60, 80% ethanol in water and after embedding in 1 wt % agarose, paraffin embedding, and sectioning, staining with Hematoxylin and Eosin (H&E) was performed and images were taken using inverted microscope with 10 and 20× magnifications.

Total Protein Content and Metabolic Activity of the Spheroids. The total amount of protein in the spheroids was measured using Pierce™ BCA Protein Assay Kit (Thermofisher). Crosslinked collagen in each spheroid was broken using 100 μL of collagenase/dispase (Sigma-Aldrich) solution (10 μL of 100 mg/mL collagenase/dispase as the stock solution in DI water, 10 μL of 10 mM CaCl₂) as enzyme activator, and 80 μL of DPBS). Half an hour later contents of each well were pipetted vigorously to break the spheroids. To deactivate the enzyme, 25 μL of 10 mM EDTA was added and incubated for 5 min. Eventually 50 μL of this solution was transferred to a new well plate and the same amount of 0.1% V/V Triton X-100 in PBS solution was added to lyse the cells with 10 min incubation in incubator. The difference between each condition and the control was reported as the total protein content of each spheroid. The same solution of enzyme and its deactivator was used as control. The same process was performed on acellular spheroids (the same collagen and medium solution in small and large wells without cells) to measure protein content of each spheroid from ECM content and eventually cell protein content of each spheroid was defined as BCA reading of spheroid with cells minus BCA reading of acellular constructs.

To measure the mass transfer in and out of each spheroids, Alamar blue assay (ABA) kit (Thermofisher) was used. Spheroids were transferred to 96 well plates and 200 μL of DMEM supplemented with 10% V/V Alamar blue solution was added to each well and incubated for 1 hr. After that, 100 μL aliquots of the medium were transferred to a black 96 well plate and reading was performed at excitation and emissions of 560 and 590 nm.

To study the reasons for the shrinkage in the spheroids, the influence of cells, transmembrane proteins and cytoskeleton were assessed. PCR was performed to study expression of cell-cell and cell-ECM junctions. For this purpose, spheroids formed in small wells with 10⁶ cells (highest cell density, S-10⁶ group) and the ones formed in large wells with 10⁵ cells (lowest density, L-10⁵ group) were chosen. Spheroids were digested the same as before and the solutions were transferred to 0.5 mL DNase free PCR tubes. Then, 250 μL PBS was added for dilution followed by centrifugal concentration of the cells and the removal of the supernatant by aspiration. To study gene expression, a one-step qRT-PCR kit (Cells-to-CT™ 1-Step Power SYBR™ Green, ThermoFisher) was used. Primers for E-cadherin (as cell-cell adhesion marker), 31-Integrin (as cell-ECM marker), and β-Actin (as housekeeping gene) were used according to Table 1. The ΔΔCt values for each primer set were calibrated to the average of housekeeping Ct values and then to the Ct values of the L-10⁵ group. For each group 4 biological replicates and 2 technical replicates for each sample was used.

TABLE 1
Sequence of the used primers for qPCR (5′ to 3′)

| Target Gene | Forward | Reverse |
|---|---|---|
| E-Cadherin | TGCCCAGAAAATGAAAAA | GTGTATGTGGCAATGCGTTC |
| | GG (SEQ ID NO: 1) | (SEQ ID NO: 2) |
| β1-Integrin | CATCTGCGAGTGTGGTGT | GGGGTAATTTGTCCCGACTT |
| | CT (SEQ ID NO: 3) | (SEQ ID NO: 4) |
| β-Actin | CATGGAGTCCTGGCATCC | ATCTCCTTCTGCATCCTGTC |
| | ACGAAACT | GGCATA |
| | (SEQ ID NO: 5) | (SEQ ID NO: 6) |

Effect of cytoskeleton on the consolidation process was studied by impairing the actin network of the cells. MCF-7 cells were cultured up to 70% confluent and pretreated with medium supplemented with 100 nM Latrunculin A (LAT-A, Abcam) for one hour. Then cells were trypsinized and spheroids were formed with 5×10⁵ cells in large wells once without LAT-A in the medium used for spheroid formation and another time with medium containing the same concentration that was used for pretreatment. The same spheroids were formed without LAT-A treatment as the control.

Spheroid Formation Using Other Cell lines. To determine whether the same technique can be used with other cell lines, large wells (4 mm in diameter) and 5×10⁵ cells in 30 μL of 1:1 collagen and DMEM solution was used with other cell lines including MDA-MB-231 and Hs-578T (two other breast cancer cell lines), SaOS-2 (osteosarcoma cell line), human umbilical cord endothelial cells (HUVEC), 3T3, L929, and Chinese Hamster ovary (CHO) cell lines (three fibroblastic cell lines), and C2C12 (myoblast cell line). All of the cells were grown in their specified culture media until 80% confluent (HUVECs were grown in EBM-2, CHO cells were grown in F12K supplemented with 10% FBS, SaOS-2 cell were grown in McKoy's media supplemented with 15% FBS, and C2C12s were grown in DMEM with 10% heat inactivated FBS. All of the other cells were grown in DMEM supplemented with 10% FBS) and trypsinized to prepare the cell suspension that were used to form spheroids. The same procedure as described previously was used to form spheroids. In all cases DMEM supplemented with 10% FBS was used for spheroid fabrication to eliminate effect of medium composition on collagen crosslinking/gelation and shrinkage pattern. All of the cell lines were acquired from ATCC©, HUVECs were used under passage number 10, and as for the rest of the cells, passage numbers below 30 were used.

Effect of cell type on mechanical properties of the spheroids was studied using a microscale mechanical test system (MicroSquisher, Cell Scale). A 3×3 mm stainless steel platen connected to a 0.4 mm diameter cantilever was pressed on the spheroids at the rate of 10% strain per minute in a displacement-controlled setup. Location of platen was tracked using a camera and a load cell connected to the other end of cantilever measured the force exerted by the spheroids. The force-displacement data were then used to measure stiffness of the spheroids. Eight spheroids (5×10⁵ cells in Large wells) were tested for each condition.

Heterogenous Multi-cellular Spheroid Formation. To determine whether this method is capable of forming heterogeneous spheroids with more than one cell type, spheroids were fabricated using bioinks consisting of MCF-7 cells along with either green fluorescent protein (gfp) tagged 3T3 fibroblasts or red fluorescent protein (rfp) tagged HUVECs in large wells. The total cell population was kept at 5×10⁵ with 90% of the cells being MCF-7 and 10% of the second cell type. Bright field images as well as fluorescent ones were taken the same as before to study effect of second cell type on spheroids shrinkage and distribution of different cell types. These results were compared to spheroids formed in the same condition but just with MCF-7 cells.

Effect of Extracellular Matrix on Spheroid Formation. Effect of the ECM type on the construct formation process and that of the concentration of the ECM was studied in large wells using collagen or Geltrex™ (ThermoFisher) as ECM. Two concentrations of bioinks were prepared by adding either 15 or 30 μL of the ECM, with 15 μL DMEM or without it, respectively, loaded with 5×10⁵ MCF-7 cells. The rest of the fabrication process was the same as that described previously.

Homogeneous Non-spherical Structures Using MCF-7. Versatility of the technique to form non-spheroidal tissue like constructs was shown using molds with different shapes. Molds in the shape of a cross (2(L)×2(W)×2(H) mm), a dumbbell (1.5 mm in radius with 3 mm distance between wells, 2 mm deep), and a series of cuboids (2(L)×2(W)×2(H) and 4(L)×2(W)×2(H) mm) were made in PDMS. Bioink was deposited into the molds and the construct allowed to assemble using the same procedure as described previously. In case of the cross structure, 10⁶ cells with total solution of 50 μL were deposited into the mold. Similarly, in the case of the dumbbell, 60 μL of the bioink containing 10⁶ cells was deposited while for the cuboid shapes 8 and 16 μL of bioink containing 5×10⁵ and 10⁶ cells were used. The ratio of collagen to DMEM was 1:1, which was the same as previous experiments. Six hours later, the constructs formed in the shape of cross were transferred to 48 well plates and images were taken 24 hrs, 3 and 7 days later to confirm their ability in maintaining their predefined shape. To show whether constructs will be able to maintain this predefined shape independent of the mold, constructs with the shape of cross were kept in 48 well plates and images were taken after 1, 3, and 7 days in each condition.

Heterogeneous Multi-cellular Non-Spherical Structure Formation. To demonstrate the capabilities of this method to fabricate heterogeneous tissue constructs molds in the shape of a dumbbell was used. Three different bioinks were loaded into different locations on the mold. Specifically, 25 μL of the bioink with 5×10⁵ of gfp-3T3 cells was added to the left well and a similar volume with the same concentration of rfp-HUVECs was added to the right well. The high viscosity of the bioink prevented its spread and spatially confined it to the round chambers into which they were deposited. Finally, 10 μL of bioink with 2×10⁵ MCF-7 cells dyed with blue cell tracker (CMF₂HC Dye, ThermoFisher) was added to the connecting channel region. Bright field and fluorescent images of the 3D tissue construct that self-assembled were taken using a stereo microscope and a ChemiDoc™ MP imaging system (Bio-Rad), respectively. After 4 hrs, close-up images of the interface regions between the different cell types were taken using an upright fluorescent microscope, in order to determine cell distribution and the shape of the tissue structure formed in these regions.

Data Analysis. Data is reported as Mean Standard Deviation (SD), statistical analysis is performed using the two-way student's t-test with an accepted statistical significance of P-value<0.05.

### Example 2. Characterization of Tissue Constructs

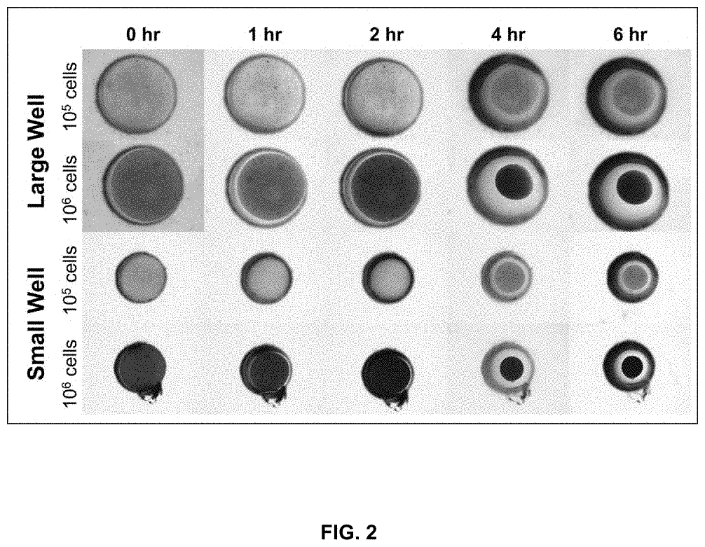

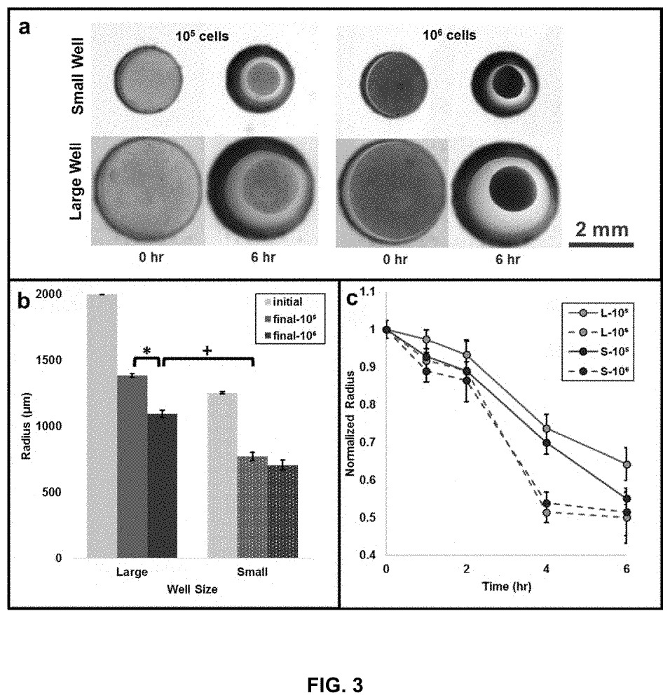

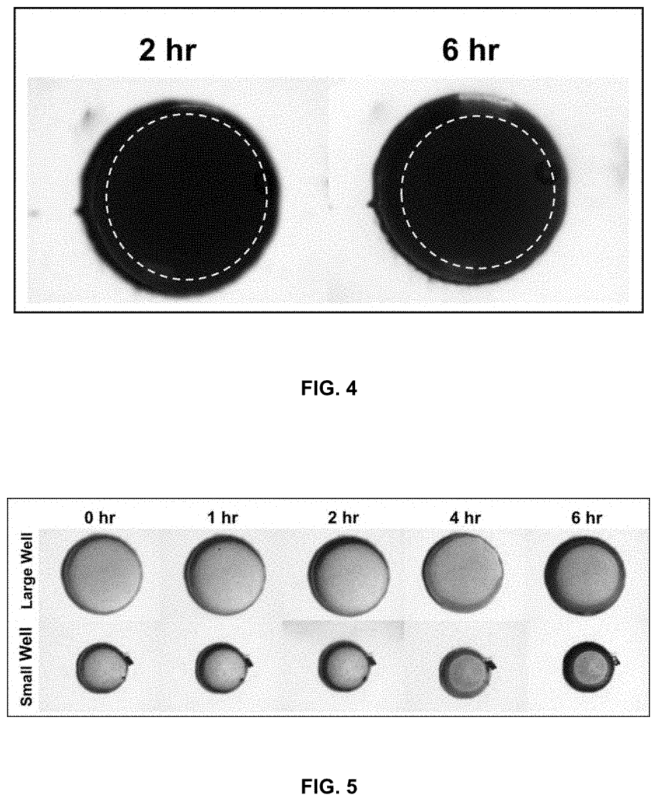

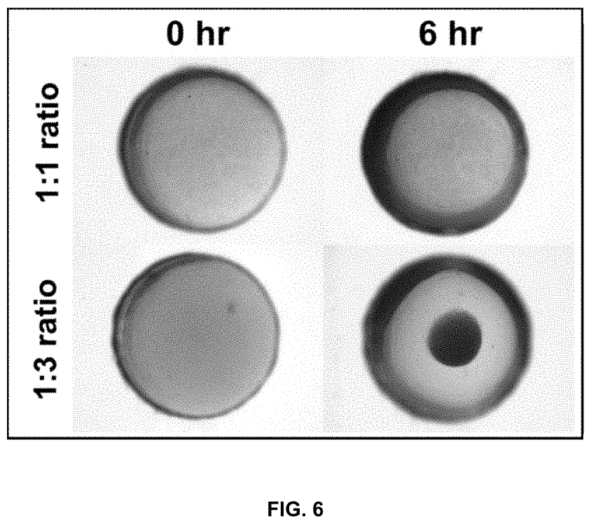

Rapid Formation of Spheroidal Tissue Constructs. In order to determine the speed with which tissue constructs are formed, bioinks were loaded into molds with circular wells and spheroidal bottom. Bioinks with various population of cells (10⁵ and 10⁶) were loaded into wells of different sizes (2.5 and 4 mm in diameter) and imaged periodically over 6 hrs (FIG. 2). For the first 2 hrs after loading, the primary mechanism of assembly was the collagen crosslinking/gelation which led to a small amount of consolidation and initial formation of the construct. The change in pH due to the addition of NaOH causes onset of collagen crosslinking/gelation that initiates the assembly process. Subsequent addition of the growth medium leads to a more dramatic consolidation with a reduction in volume (-70% when 10⁶ cells were used and 50% in case of 10⁵ cells independent of well size) as shown in FIG. 3. The second phase of consolidation does not happen in the absence of cells (FIG. 4) indicating the key role played by the cells in this process. Bioinks loaded into both large and the small wells were consolidated, as shown in FIG. 3b, in a highly repeatable manner with very small variation in the sizes (3-6% in all cases), unlike many of the other spheroid generation methods. The final consolidated volume of the tissue construct was not dependent on the initial population of cells for small wells while it was significantly different in the case of large wells. It is interesting to note that the trajectory of the consolidation is only dependent on the cell population in the wells and not on the size of the wells as shown in FIG. 3c. The construct formed was found to be mechanically robust and easy to handle after 6 hrs unlike other methods [1] where the consolidation process takes several days (up to 7) for similar amount of consolidation. This method is scalable and able to form spheroidal constructs with cell population as low as 50,000 cells in both small and large wells (FIG. 5). Changing the ratio of the collagen to DMEM loaded with cells (5×10⁴ cells in large wells) in the bioink from 1:1 to 1:3 resulted in smaller spheroids (FIG. 6) demonstrating the role of ECM in determining the final consolidated size.

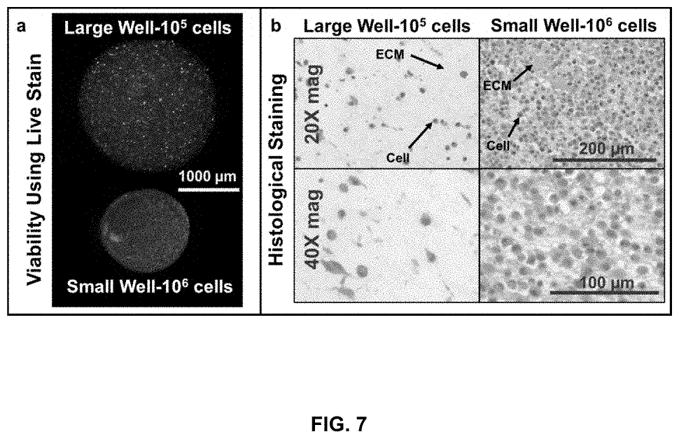

The distribution of live cells within the spheroid formed was determined using live cell staining with calcein-AM (FIG. 7a). It shows that at the end of the process there was a uniform distribution of live cells in all regions of the spheroids. Spheroids formed in large well with 10⁵ cells and small well with 10⁶ cells were chosen for live staining as the first one has the largest size with lowest cell density while the latter is the smallest with highest cell density. Interestingly, the cells at the center of the spheroid appear to be alive and with the same density as those at the surface even though the diffusion limit in the spheroids is similar to avascular tissues and is around 150 to 200 μm. This is probably due to the fact that the fabrication process is very fast and viability of the cells is not affected during this time period.

The spheroid fabrication process can also be used to control the primary type of interaction of the cells. A high density of cells in the spheroid will promote a greater cell to cell interaction while a lower density will provide more cell ECM interactions. In order to demonstrate this, the same spheroids for live staining were used and histological staining was performed using H&E to show the distribution of the cells in each condition (FIG. 7b). They show that it is possible to create conditions where the cells are closely packed and cell-cell interaction is substantial by using small wells and high cell numbers. Similarly, for those assays that investigate cell-ECM interactions, large wells with low cell numbers would be suitable.

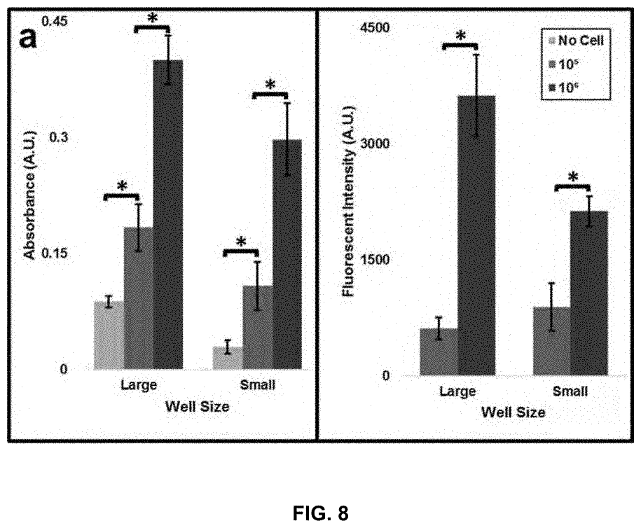

Total protein content of each of the spheroids was measured and compared to each other and acellular spheroids using Pierce BCA kit (FIG. 8a) and as expected this amount is dependent on both well size which represents the amount of collagen used and the total cell number in each spheroid. Comparing spheroids with the same cell number but different well sizes shows that total protein is significantly dependent on the cell number less on the well size. FIG. 8b represents the total metabolic activity of each of the spheroids using Alamar blue. Interestingly, metabolic activity changed much more significantly with cell numbers in large wells as compared with smaller wells. This may be related to transport dynamics of the Resazurin sodium salt and the final product in and out of the spheroids which is related to the compactness of the structure as well as their radii. The ratio of cell protein content of spheroids formed with 10⁶ to the ones with 10⁵ cells is 3.26 and 3.40 in large and small wells, respectively, while ratio of their metabolic activity is 5.90 and 2.38. Based on these results although BCA readings show the same increase from 10⁵ to 10⁶ cell number in each well size, the ABA readings are not following the same pattern which indicates importance of spheroid size and compactness in mass transport properties and thus careful control of the well size and the cell numbers can be used to modify transport dynamics of drugs in and out of the spheroids formed with applications in drug screening.

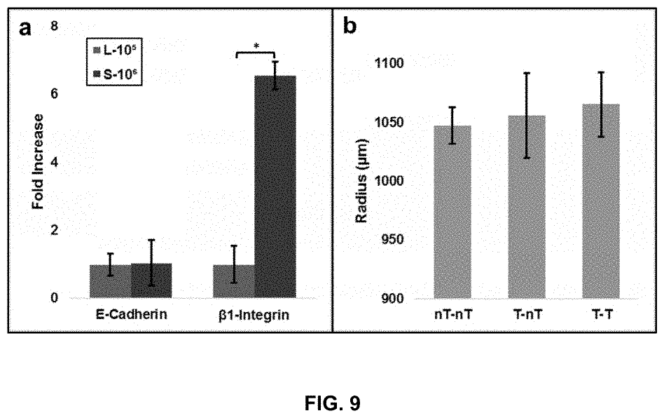

The microstructure of the spheroids was also studied. Considering the final sizes of the spheroids and cell numbers in each of them, it was expected that spheroids with 10⁶ cells formed in small wells had higher densities. As it is shown in FIG. 9a, while there is no meaningful difference between expression of E-cadherin, a cell-cell junction protein, in the two studied groups (spheroids with higher cell density and spheroids with lowest density), 0-integrins, cell-ECM junctions, are expressed more than 6-fold higher in denser spheroids (S-10⁶) which explains the higher amount of shrinkage observed. It has been shown that formation of multicellular spheroids includes an initial phase of integrin-ECM interaction to form the aggregates which is then followed by the enhanced cell-cell interactions through cadherins which causes the final compaction. The different timeframe of action between these two phases can be due to the time needed for expression of sufficient amount of E-cadherins on the cell membrane. Formation of a spheroid starts by formation of loose aggregates with initial cell-ECM attachments which later forms a compact solid structure by accumulation of cadherins on cell surface and their hemophilic binding.

To study whether cell-cell and cell-ECM adhesions or contraction caused by cytoskeleton are dominant in spheroid formation, MCF-7 cells were treated with actin cytoskeleton influencing drug latrunculin A (Lat-A) that is known to bind to actin monomers and prevent their subsequent reorganization of cytoskeleton in MCF-7 cells. Spheroids were formed with 5×10⁵ cells in large wells without pre- or post-treatment (nT-nT), pre-treated cells with 100 nM for 1 hr without post-treatment during spheroid formation (T-nT), and pretreated cells with post-treatment using the same concentration as pretreatment step (T-T). Spheroids were formed in all cases and there was no meaningful difference between their radii after 6 hrs (P-value>0.05) which combined with increased expression of integrins shows the importance of cell-cell and cell-ECM interactions over cytoskeleton remodeling and reorganization during the first few hours of the shrinkage process (FIG. 9b). Such multifactorial effects on cell aggregation have also been observed in aggregation of cell types without the ECM present, where cytoskeleton tension and cell-cell adhesion play an opposing role in determining the extent of compaction observed.

### Example 3. Multicellular Spheroidal Tissue Constructs

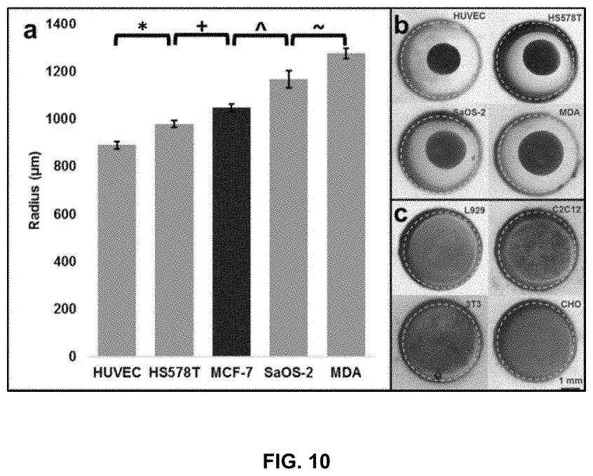

Homogeneous Multi-cellular Spheroid Formation. The ability of different cell types to rapidly form spheroids was evaluated by using eight other cell lines (in large wells with 5×10⁵ cells). Some of the cell types such as HUVEC and HS578T demonstrated a higher propensity for rapid consolidation as compared with MCF-7 cells while others such as SaOS-2 and MDA demonstrated a lower propensity as shown in FIG. 10a. For instance, the spheroids made from HUVEC cells consolidated to a radius of 890.23±15.48 μm within 6 hrs from the original well with 2 mm in radius while spheroids made using L929 bioink hardly reduced in size within the given timeframe (FIG. 10b) and only had the shrinkage associated with the original collagen crosslinking/gelation in the first phase of consolidation. C2C12, 3T3, and CHO cells didn't show any shrinkage either. This propensity may be linked to the differing ability of the cell types to express adhesion molecules such as cadherins or integrin within the short time frame.

Multicellular spheroids and other self-assembled constructs have been used for different applications such as modeling naturally occurring processes, as a model for cancer research and drug discovery, as well as building blocks in tissue engineering. One of the limitations of using these as building blocks for large tissue constructs is diffusion limitations and the need for vascularizing the structure.

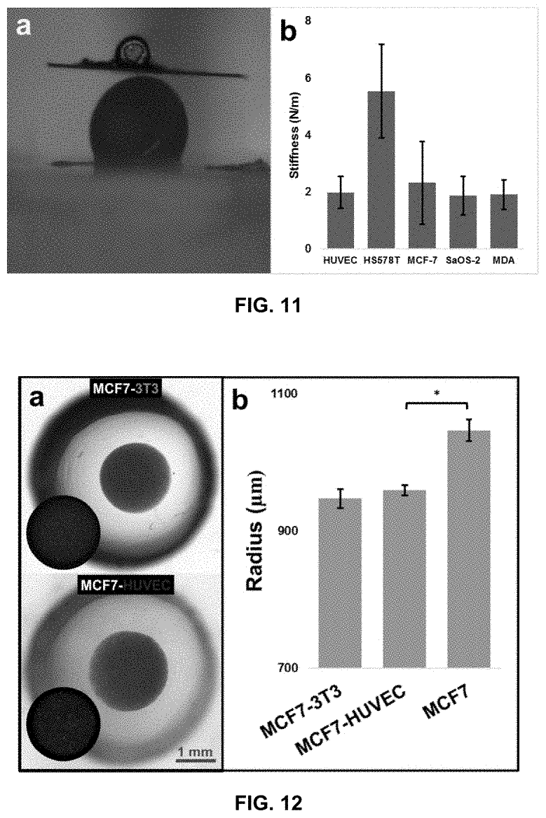

Using MicroSquisher testing machine (setup shown in FIG. 11a) spheroids formed above were tested under compression and their compressive stiffness is calculated as the slope of the initial linear region of Force-Displacement diagram (FIG. 11b). Based on these results although spheroids formed with different cells showed different amount of compaction, the final stiffness is the same for most of them once their initial cell density and well size were the same. The only exception was the human carcinosarcoma cell line which was observed to have a significantly different stiffness as compared with other cell lines. Collagen constructs without cells formed with the same conditions didn't have enough mechanical stability and disintegrated in the process of transferring them from the PDMS wells to the Microsquisher machine. These preliminary results indicate that the cell-ECM interaction is involved in the formation of mechanically stable spheroids.

Heterogeneous Multi-cellular Spheroid Formation. Ability of the method to form homogeneous multi-cellular spheroids was demonstrated by using a bioink that consisted of 90% MCF-7 cells along with 10% of 3T3s or HUVECs. These specific cell types were chosen as interaction of stromal cells such as fibroblasts and endothelial cells with cancer cells is an active area of study and developing 3D models that can recapitulate these interactions is important.

The total cell population was fixed as 5×10⁵ cells and spheroid formation occurred in large wells. All of these bioinks were found to result in spheroid formation as shown in FIG. 12. Bright field and fluorescent images shown in FIG. 12a also demonstrate that the cells are uniformly distributed within these spheroids. Interestingly, addition of a small percentage of cells of the second type caused additional consolidation of the spheroids even when some of the cell types (3T3s) didn't not cause consolidation by themselves within the given timeframe. For instance, bioinks with MCF-7 cells alone consolidated to form spheroids that were 1047.17±15.6 μm in size but replacing of 10% of cell population with HUVECs resulted in further decrease in the final size to 959.30±7.8 μm. Using 3T3s that by themselves did not cause considerable consolidation of the spheroids also produced a spheroid of the size of 947.28±13.3 μm with higher shrinkage compared to MCF-7 ones alone which could be because of the increased expression of E-Cadherins in cancer cells when co-cultured with fibroblasts which as an epithelial adhesion molecule plays an important role in compaction of the cells in the process of spheroid formation.

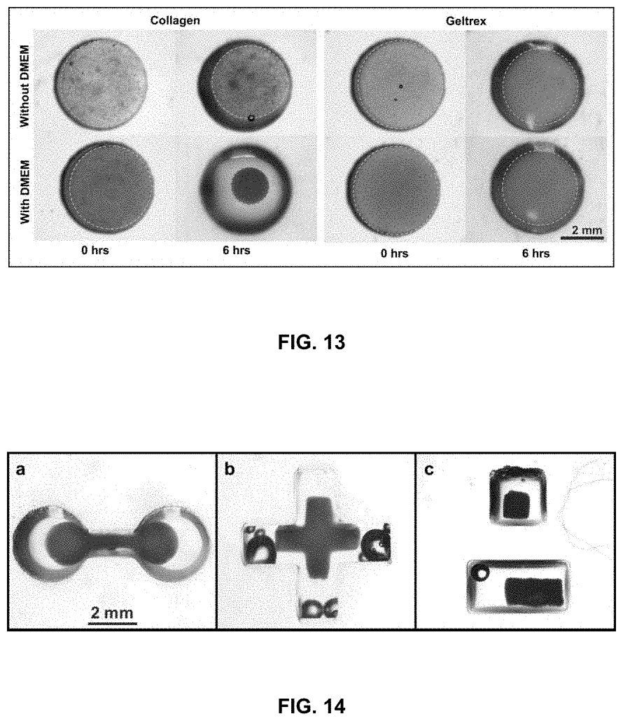

Effect of Extracellular Matrix on Spheroid Formation. Previously, it was determined that cells were essential for rapid formation of the tissue constructs. In order to determine other essential conditions, the method was tested with different ECM. Apart from collagen many other natural ECMs such as laminin, elastin, glycoproteins and proteoglycans are also looked upon as important for 3D culture and to recreate the tissue microenvironment. Matrigel™ and its reduced growth factor version, Geltrex™ are some of the most widely used examples of such ECMs. The use of Geltrex™ was evaluated with this method and also investigated the impact of the second addition of DMEM in the consolidation process. FIG. 13 shows the grafts formed with collagen and Geltrex™ with and without DMEM. As it can be seen only in the case of collagen with DMEM was the consolidation significant. This experiment indicates that collagen and the addition of DMEM 2 hrs into the spheroid formation process is important for the rapid consolidation. In all of the scenarios, it was found that the ECM crosslinked in less than 2 hrs and formed a solid structure (in case of collagen by adjusting its pH to 7.4 and in case of Geltrex™ by increasing the temperature). Once the pH of the collagen solution is increased to 7.4 it starts to exclude water and self-assemble into crosslinked fibrils forming well connected scaffold. Furthermore, cells have transmembrane adhesion receptors that adhere to collagen, enabling forces to be transmitted from the cells onto the matrix scaffold. This coupling between the ECM and the cells promotes the consolidation process. The exclusion of water pushes it out of the scaffold and promotes consolidation. Geltrex on the other hand does not precipitate out and therefore will not exclude as much water from the matrix as collagen, leading to a considerably less consolidation. The addition of DMEM is also essential as it provides additional growth media which enables the cells to attach to the ECM and exert forces required for consolidation.

### Example 4. Homogenous and Heterogeneous Non-Spherical Constructs

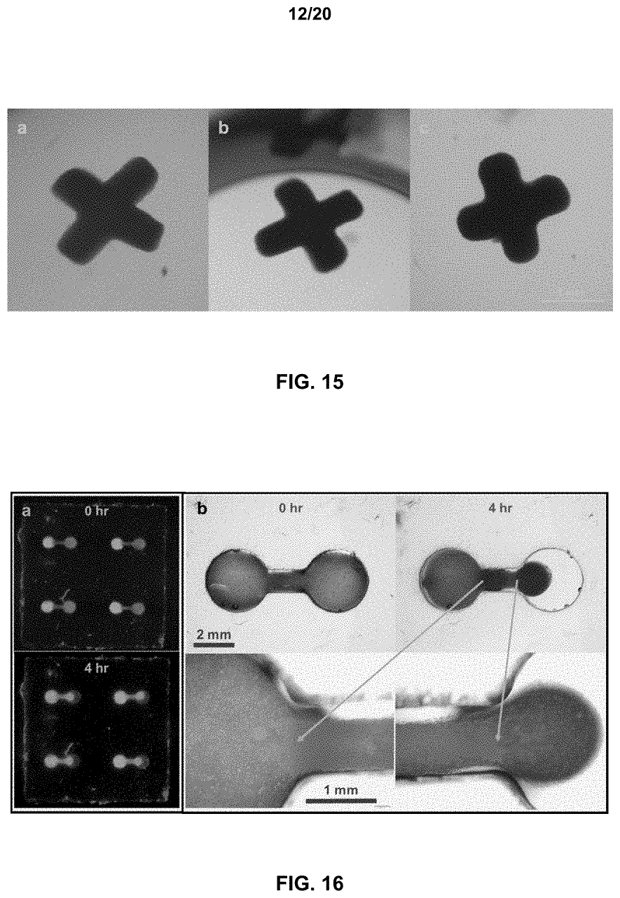

Homogeneous Non-spherical Structures Using MCF-7. Multicellular spheroids are widely used because of their ability in resembling structure of real tissues and conditions such as initial avascular state of tumors, but the formation of a necrotic core is not preferable in the study of tissue types and biological systems where non-spherical constructs can be used. To this end, molds with different shapes were used with the same process as before in order to determine whether initial shape at which collagen crosslinks/gels defines the final shape or the forces exerted by the cells during the shrinkage process. FIG. 14 represents the final shape of these non-spherical aggregates. The tissue constructs consolidated isotopically in all directions after the initial collagen crosslinking/gelation phase that fixed the shape of the initial scaffold structure. These structures could be removed after 6 hrs from the molds whereupon they acquire enough structural stiffness and stability to be handled with tweezers. Interestingly, these structures retained their shape even when unconstrained as shown in FIG. 15 where they were kept in a 48 well plate for a further 24 hrs, 3 and 7 days. Though non-spherical cell-embedded collagen aggregates has been prepared before using PDMS molds for different applications [2-4], these constructs required micro-cantilevers in the molds formed using a multistep photolithography process to hold the consolidating construct to the non-spherical shape. In the absence of such constraints these constructs would revert to the spherical shape. Unlike other techniques, the method of the present application does not require complex mold fabrication or cantilevers and utilizes the two-step consolidation process to fix the shape of the scaffold and then introduce isotropic consolidation. In addition, the constructs disclosed herein can be physically removed from their molds easily for further processing, while those attached to the cantilevers are fixed in place. Other approaches of tissue construct formation using collagen [1] in PDMS molds are not rapid (take several days as compared to 6 hrs) and were not able to maintain their initial shape once removed from the mold unlike the constructs of the present application (FIG. 15). The two-step process for consolidation and the use of higher concentration of collagen (5 mg/mL vs. 3 mg/mL) are important for the difference in the observed consolidation process. The ability to allow the sequenced assembly of the collagen into fibrils and then subsequently promote the rapid binding and force exertion of the cells using addition of DMEM after 2 hrs leads to formation of tissue constructs of any shape which is fixed even when unconstrained that opens significant possibilities for engineering them.

Heterogeneous Multi-cellular Non-Spherical Structure Formation. In natural tissues different cell types are positioned adjacent to each other in close proximity. The signaling from one cell then affects the behaviour of the neighboring one and helps to recreate an in vivo-like microenvironment. The ability of this method to form multicellular structures with predefined positioning of cells was shown by formation of multi-cellular dumbbells. FIG. 16 represents the bright field and fluorescent images of the multicellular-dumbbells after 0 and 4 hrs with three different cell types in left, center, and right sections of the structure. Even though the bioinks have been introduced into the different regions of this mold one after the other, the consolidation process is smooth and a single continuous construct is formed (FIG. 16a). There is also not much mixing between the regions and a clear and intimate interface is formed where different cells are positioned at different locations within the same ECM. It is also interesting that due to the different cell types used in different locations the amount of consolidation that occurs varies. Nevertheless, that does not cause separation and a smooth continuous construct is formed to accommodate all the different stresses imposed by the cells. FIG. 16b shows this continuity and defined distribution of cells according to the initial pattern of the mold even after they have shrunken to their final size and structure. Structures like this can be used to study indirect effect of cells on each other, for example through paracrine activity. Unlike other methods for formation of heterogeneous constructs multiple cells [5] that require restraining features to form non-spherical structures and several days for the different cell types to grow towards each other to achieve intimate contact, this method is rapid and extremely precise in spatial positioning. It is also capable of retaining the established non-spherical geometry even when removed from the mold for further processing.

### Example 5. Tubular Constructs and Physical Stimuli

Current 3D models lack either the rich multicellular environment or fail to provide appropriate biophysical stimuli both of which are required to properly recapitulate the dynamic in vivo microenvironment of tissues and organs. This is because many of the current techniques used for making these constructs are limited in the cell density, fabrication speed, control over positioning of different cell types, and creation of tissue/organ interfaces. More importantly, formation of necrotic cores and the inability to grow them beyond a certain size due to mass transport limitations is one of the key limitations of multicellular spheroid models, especially for applications other than modeling avascular stage of the cancerous tissues. Finally, it is also difficult to incorporate biophysical cues such as electrical and mechanical stimulation that is increasingly being considered important to recreate the in vivo environments, due to their form factor.

In summary, existing methods do not combine all the required features including rapid self-assembly of 3D tissue constructs, ability to precisely position different cell types and pattern them, ability to scale sizes of the constructs, and the ability to incorporate all the three important biophysical stimuli, stretch, shear, and electric. More importantly, they also involve customized molds, tools and equipment that make it difficult to implement without appropriate engineering expertise.

In this example, the rapid construction of multicellular, tubular tissue constructs termed “Tissue-in-a-Tube” using self-assembly process in tubular molds with the ability to incorporate a variety of biophysical stimuli such as electrical field, mechanical deformation, and shear force of the fluid flow is described. Unlike other approaches, this method is simple, requires only oxygen permeable silicone tubing that molds the tissue construct and thin stainless-steel pins inserted in it to anchor the construct and could be used to provide electrical and mechanical stimuli, simultaneously. The annular region between the tissue construct and the tubing is used for perfusion. Highly stable, macroscale, and robust constructs anchored to the pins form as a result of self-assembly of the ECM and cells in the bioink that is filled into the tubing. Patterning of grafts containing cell types in the constructs in axial and radial modes with clear interface and continuity between the layers is demonstrated. Different environmental factors affecting cell behavior such as compactness of the structure and size of the constructs can be controlled through parameters such as initial cell density, ECM content, tubing size, as well as the distance between anchor pins. Using connectors, network of tubing can be assembled to create complex macrostructured tissues (e.g. centimeters length) such as fibers that are bifurcated or columns with different axial thicknesses which can then be used as building blocks for biomimetic constructs or tissue regeneration. This technique is simple (no microfabrication steps required) and fast (only a few hours culture time before stable tissue constructs are formed) without the need for specific fabrication equipment, has the ability to control positioning of multiple cell types/ECM materials with uni- or multi-directional crosstalk between them. The method is also versatile and compatible with various cell types including endothelial, epithelial, skeletal muscle cells, osteoblast cells, and neuronal cells. As an example, long mature skeletal muscle and neuronal fibers as well as bone constructs were fabricated with cellular alignment dictated by the applied electrical field. The versatility, speed, and low cost of this method is suited for widespread application in tissue engineering and regenerative medicine.

Thus, large constructs with cylindrical shape, and uniform and well-defined mass transport properties without necrotic cores are created with high cell density, with multiple cell types positioned in predefined patterns and with clear interfaces, combined with multitude of electrical/mechanical stimulation to create a dynamic environment. The cylindrical format enables scalability and construction of various sizes (e.g. mm to cm). The format inherently is suited for perfusion of media to support metabolic needs of cells and create biomimetic shear conditions resulting in a physiologically relevant model that very closely mimics the in vivo conditions. Macrostructures with different shapes can also be used as cell vehicles for implantation or as in vitro models for applications such as drug screening.

### Materials and Methods

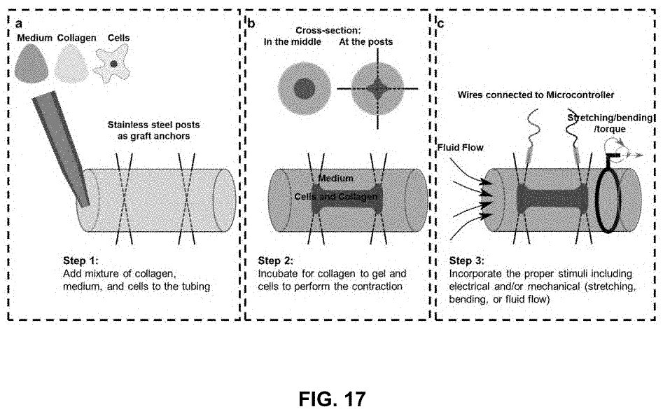

Collagenous constructs of cells inside a silicone tubing and anchored to the metallic pins were formed using a process of self-assembly that has been used previously for spheroidal and non-spheroidal structures. Replacing PDMS molds with silicone tubing enables production of solid tube-like constructs which are anchored on the stainless-steel pins (FIG. 17). The silicone tubing is widely available, do not require any special fabrication process and can be connected using connectors to form complex networks. It is also gas permeable. The pins can be inserted into the tubing without leakage and serve as anchoring points axially to shape the construct formation and to apply axial tension as the construct forms.

Once the self-assembly process is complete an annular gap forms between the construct and the tubing that can be used for perfusion purposes and to apply shear forces on the construct. The inserted pins can be connected to a microcontroller to apply electrical stimulation to the construct, and the tubing itself can be stretched, bent, or torqued to create different types of mechanical deformation in the construct. This setup allows formation of collagenous constructs in a very short process (4-6 hrs) that can be kept and monitored in a true 3D and dynamic environment with different types of stimuli.

Cell Culture. Different types of cells were used in the current study for different purposes. Michigan Cancer Foundation-7 (MCF-7) breast cancer cells were cultured in Dulbecco's Modified Eagle Medium (DMEM) (with L-glutamine and high glucose, Gibco), supplemented with 10% V/V fetal bovine serum (FBS) (Canada origin, Thermofisher) and 1% V/V Penicillin-Streptomycin (10,000 U/mL, Thermofisher) until 70% confluent. C2C12 myoblast cells were grown in the same DMEM, supplemented with 10% V/V heat inactivated FBS (HI-FBS) (Canadian origin) and 1% V/V Penicillin-Streptomycin. For differentiation purposes, these cells were cultured in DMEM supplemented with 2% V/V of horse serum (Thermofisher) and 1% V/V Penicillin-Streptomycin and 0.1% Insulin (Insulin-Transferrin-Selenium, 100×, Thermofisher, Catalogue number 41400045). SH-SY5Y neuroblastoma cells were cultured in DMEM/F-12 (Thermofisher, with L-glutamine) medium supplemented with 10% HI-FBS and 1% Penicillin-Streptomycin. For differentiation of these cells DMEM/F12 was supplemented with 1% heat inactivated FBS, 1% N2 supplement, and 1 M retinoic acid. Red fluorescent protein (rfp)-tagged human umbilical vein endothelial cells (HUVEC) were grown in EBM-2 medium. Osteoblast-like cells from Saos-2 osteosarcoma cell line were cultured in McCoy's medium (Thermofisher, with L-glutamine) supplemented with 15% FBS and 1% Penicillin-Streptomycin.

Tissue-in-a-Tube: Fabrication and Optimization. MCF-7 cells were used for characterization purposes to study effect of collagen to medium ratio (CMR), cell density, tubing size, and distance between the stainless-steel pins. 1:1, 1:3, and 1:5 ratios were used while other parameters were kept constant at 2×10⁶ cells/mL, tubing with 3 mm inner diameter (ID), and pins being 2 cm apart. The 1:1, 1:3 and 1:5 ratios corresponded to 2.5 mg/ml, 1.25 mg/ml and 1 mg/ml of effective collagen concentration in the final solution. Effect of cell density was studied by using bioinks containing 1, 2, and 3×10⁶ cells/mL of the bioink with 1:3 CMR and 2 cm wide pins in 3 mm ID tubing. Effect of Tubing size was studied by using tubing with 1, 3, and 7 mm ID, termed as thin, medium, and thick, respectively, while 1:3 CMR, 2×10⁶ cells/mL bioink, and 2 cm wide pins were used. In order to study the ability to form constructs with different lengths, tubing with 3 mm ID was used with a bioink with 1:3 CMR and 2×10⁶ cells/mL density but pins were kept 2 and 4 cm apart. After filling the tubing with the bioink in each case, incubation at 37° C. was performed for 4 more hours until shrinkage of the stable constructs was done. Images of the samples were taken using a dissecting microscope (Infinity Optical Systems). Bioink was prepared by dispersing cells in the required volume of the medium and then addition of neutralized bovine collagen I (Thermofisher, 5 mg/mL). Collagen was neutralized by addition of 0.1 M sodium hydroxide in DI water. Stainless steel 304 wire (McMASTER-CARR) with 0.5 mm diameter were used as pins and at each point two pins perpendicular to each other were inserted in the tubing to provide proper anchorage for the constructs.

Live/dead staining was performed using the kit (ThermoFisher) following the provided protocol. Briefly, calcein-AM and ethidium homodimer-1 were diluted in the medium and added to the samples (formed with 1:3 CMR and 2×10⁶ cells/mL density in tubing with medium thickness and with 2 cm apart pins) 4 hrs after process was started followed by 1 hr of incubation. Images of the samples were taken using an inverted fluorescent microscope with 4× magnification and proper filters.

Controlled Cellular Interfaces. Formation of clear and continuous interface between regions containing different cell types in a contiguous tissue construct was shown in both axial and radial configurations. MCF-7 cells were stained with either green DiO or red DiI fluorescent cell trackers (Thermofisher). For the axial configuration, half of the tubing was filled with the bioink containing green stained cells (1:3 CMR, 2×10⁶ cells/mL solution). After half hour incubation when the collagen had gelled but the cells had not attached to the ECM to apply significant traction forces, the other half of the tubing was filled with the same bioink but with red stained cells. For radial configuration, the whole tubing was filled with green stained cells' bioink (1:3 CMR, 2×10⁶ cells/mL solution). After 2 hrs of incubation that shrinkage was performed, extra medium was extracted and a 1:3 CMR bioink with 1×10⁶ cells/mL was added followed by further incubation. Fluorescent images were taken before and after addition of each bioink using a ChemiDoc™ MP imaging system (Bio-Rad).

Complex Macrostructures. Macrostructures with different patterns including bifurcated patterns and columns with varying axial thicknesses were formed using HUVECs. For bifurcated patterns, three 3 mm ID tubing, each 2 cm in length were connected to each other using a Y-shaped connector. At the end of each tubing two perpendicular pins were inserted as anchor pins and the entire connection was filled with 1:3 CMR and 2×10⁶ cells/mL solution of HUVECs. After 1 hr of incubation that collagen had gelled, and some shrinkage was observed, pins were removed and bifurcated macrostructure was retrieved from the tubing. Columns with descending thicknesses were formed by connecting 2 cm long tubing with 7, 3, and 1 mm IDs using proper connectors, respectively. Perpendicular pins were inserted in the middle of each tubing and the same bioink as before was added. Macrostructure was retrieved after 1 hr of incubation. Fluorescent images were taken using the same ChemiDoc™ MP imaging system, before and after samples were taken out of the connected tubing.

Dynamic Environment. C2C12 constructs were formed with 1:3 CMR and 2×10⁶ cells/mL bioink in tubing with 3 mm ID and 2 cm apart pins in the cells' growth medium. After 24 hrs, the medium was switched to the cells' differentiation medium and at the same time a step electrical signal with peak to peak voltage of 10 V (5 V/cm) and frequency of 50 Hz was applied (5 samples in parallel). An open source microcontroller, Arduino Uno R3, was used to create this signal and to control a motor that was used for perfusion (flow rate of 0.1 mL/min for 1 min every 12 hrs). The code used for programming the microcontroller that controls the bioreactor is included in Table 1. This group of samples were named “Dynamic”. Samples were kept in this condition for 3 more days. As control groups, samples formed in the tubing for 24 hrs but retrieved from it and kept in 6 well plates in 2 mL differentiation medium (“In Well” group), and samples kept in tubing with differentiation medium but without electrical stimulation (“In Tube” group) were considered. At day 4, samples were taken out of the tubing and images were taken using the dissecting microscope used previously. ImageJ software was used to measure thickness of the constructs before and after releasing them from the anchor pins and were compared to the “In Well” samples.

Three samples for each condition (n=4) were digested using a 0.5 mL of 2 V/V % collagenase/dispase (Sigma-Aldrich) solution in PBS (stock solution was 100 mg/mL collagenase/dispase in DI water). After digestion was done another 0.5 mL of 0.5% Triton X-100 in PBS was added to lyse the samples. Pierce BCA (Thermofisher) kit was used to measure the protein content of each sample by using two 25 μL aliquots of lysate solution in 96 well plates where 200 μL of kit solution (50:1 ratio mixture of parts A and B of the kit) was added to each well. Absorbance was measured at 562 nm after 30 min incubation at 37° C. in duplicate reading for each sample. Mixture of Collagenase/dispase and Triton X-100 solutions was used as control and its value was subtracted from the samples.

Three more samples for each condition were fixed in 2% formaldehyde solution in DI water for 1 hr. After fixation was done, samples were washed with warm PBS two times and 1 mL of PBS containing 25 μL of Alexa Fluor™ 488 Phalloidin (Thermofisher) stock solution (300 units dissolved in 1.5 mL methanol) and 0.2% Tween-20 as permeabilizing agent was added with 1 hr incubation at room temperature. After washing with PBS, samples were counterstained with 1 mL PBS containing 1 μL of DAPI (4′,6-Diamidino-2-Phenylindole, Dihydrochloride, Thermofisher) stock solution (10 mg/mL in DI water) for 30 min. Imaging was performed using an inverted fluorescent microscope (Olympus, USA) with DAPI and FITC filters with Ex/Em of 381-392/417-477 and 475-495/512-536, respectively. Live/dead staining and imaging was performed as before on the “Dynamic” and “In Tube” groups after samples were taken out of the tubing at day 4.

Constructs were also formed with SH-SY5Y and Saos-2 cells in 3 mm ID tubing with 1:3 CMR and 2 cm apart pins while cell density was 4×10⁶ cells/mL for SH-SY5Y cells and 2×10⁶ cells/mL for Saos-2 cells. Differentiation of SH-SY5Y was started at day 1 by switching to their differentiation medium and in both cases electrical stimulation was started after 1 day and continued for 5 days with 10 V peak to peak and 50 Hz frequency.

Statistical Analysis. Data are reported as Mean Standard Deviation (SD) and statistical analysis was performed using one-way ANOVA test in GraphPad Prism with an accepted statistical significance (p-value) less than 0.05. Significant outlier data points were detected using Grubbs' test.

### Results and Discussion

Tissue-in-a-Tube: Fabrication and Optimization. A biofabrication approach termed as Tissue-in-a-Tube has been developed to form highly dense multicellular cylindrical constructs, rapidly with the ability to incorporate electrical and/or mechanical stimuli to cells in a 3D environment along with continuous medium perfusion (FIG. 17). Silicone tubing with stainless steel pins inserted in it were used as the molds for the assembly of 3D collagenous constructs. These pins, inserted at specific locations, act as anchors and direct the self-assembly of the constructs between them when appropriate bioinks are injected into the silicone tubing (FIG. 17a). They also allow application of electric field axially over the construct during or after its formation process. The self-assembly process leads to shrinkage of the collagenous bioink into a dense construct at the center of the tubing leaving a uniform concentric gap around it that can be used for perfusion of nutrients, removal of waste, and to apply shear stimulus (FIG. 17b). The silicone tubing is also gas permeable and thus allows gas exchange to support long term tissue culture. It is also flexible and mechanical deformation of it such as stretching, bending, or torsion can induce similar effect on the anchored tissue constructs (FIG. 17c). Single or multiple stimuli can be applied to the constructs in a time dependent manner depending on the cell types used in the biofabrication process. The technique is simple, low-cost, rapid, and can be used with a variety of cell types including epithelial (such as MCF-7 breast cancer cells) and endothelial (such as HUVECs) cells, skeletal muscle cells (such as C2C12 cells), neuronal cells (such as SH-S5Y5 cells), and bone cells (such as Saos-2 cells), either alone or in co-culture as shown in detail in the following sections. It can also form larger constructs with multitude of stimuli applied simultaneously.

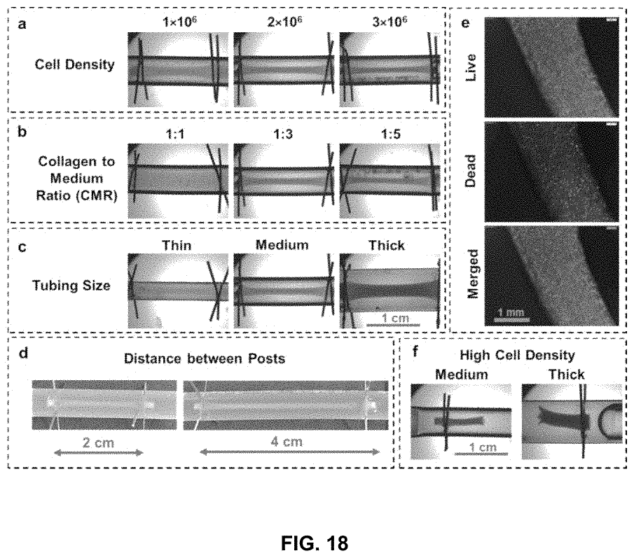

Different parameters such as cell density, initial collagen to medium ratio (CMR), and tubing size (inner diameter (ID)) can affect the dimensions of the formed construct as well as its compactness which was characterized using MCF-7 cells with the epithelial phenotype characteristic. Increasing the cell density and CMR, or decreasing the tubing ID, decreased the thickness of the construct (FIG. 18a-c). For instance, increasing the cell density from 1 to 2 and 3×10⁶ cells/mL while the CMR and tubing ID were kept at 1:3 and 3 mm, decreased the diameter of the tubular graft from 1854±45 to 1375±41 and 102851 m, respectively (n=4). It should be noted that the formation of the construct and the contraction leads to a dramatic increase in its cell density (final densities of 0.28×10⁷, 1.06×10⁷, and 2.54×10⁷ cells/mL for initial densities of 1, 2, and 3×10⁶ cells/mL, respectively, when CMR and tubing ID of at 1:3 and 3 mm were used). Starting with a higher cell density will also result in a higher relative increase in the final construct as more shrinkage happens due to higher cell-cell interactions. For example, the total increase in the final density of constructs with seeding densities of 1, 2, and 3×10⁶ cells/mL was 2.8, 5.3, and 8.5, respectively. Thus, different volume ratios of collagen to medium, cell densities, and tubing sizes can be used to change the compactness and size of the construct.

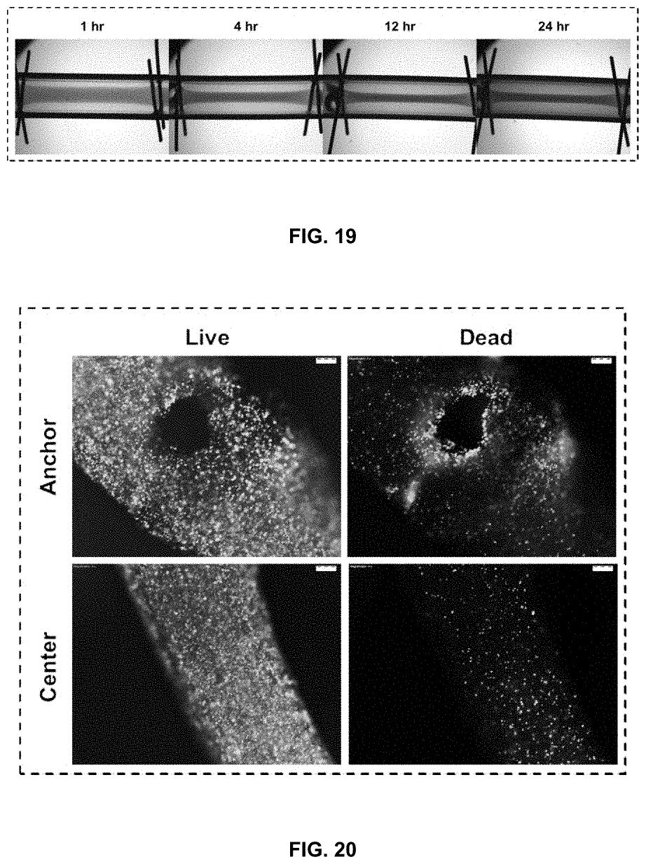

Increase in cell density leads to increase in cell-cell and cell-ECM interactions that facilitate higher traction forces and increased consolidation of the construct. Longer constructs were formed by increasing the distance between the anchor pins while maintaining the diameter of the tubular structure (FIG. 18d). The rate of contraction due to self assembly is dramatic in the first 4-6 hrs and decreases over the next 20 hrs (i.e. much lower shrinkage) after which the size of the construct stabilizes (FIG. 19).

Bioinks with CMR of 1:3 and cell density 2×10⁶ cells/mL seeded into tubing with anchor pin spacing of 2 cm and 4 cm produced correspondingly long constructs with minimal change in their diameter (1375±41 vs. 1320±89 m for 2 and 4 cm apart pins, respectively). Since construct formation process is fast (4 hrs), the cells were viable and only a small number of dead cells can be observed with uniform distribution rather than formation of necrotic regions (FIG. 18e). Interestingly, the number of dead cells close to the anchoring pins was slightly higher than in the rest of the construct (these other regions of the construct showed a uniform distribution of live cells with only a low number of dead cells) which could be due to higher traction forces in those regions (FIG. 20). Increasing the cell density increased the amount of internal strain generated in the construct resulting in excessive contraction which led to its catastrophic failure (FIG. 18f).

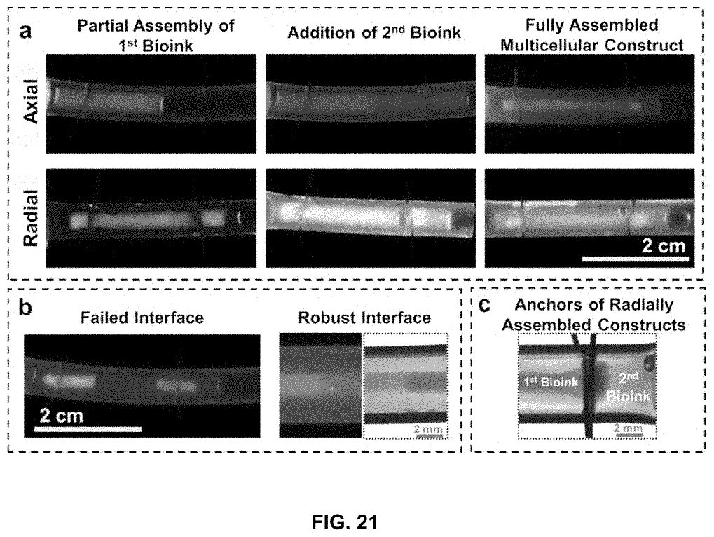

Controlled Cellular Interfaces/Complex Macrostructures. Multilayered and multi-material tissue engineered constructs better mimic function and architecture of natural tissues. Such constructs can be used to study the interaction between different cells in a tissue that happens through paracrine or contact-dependent cell signaling which significantly influences their individual function. The rapid self-assembly process used in this method enables formation of constructs that can be axially or radially patterned with different cell types (demonstrated here with MCF-7 cells stained with different colors) while maintaining its structural continuity and integrity (FIG. 21a). In order to be able to produce axial patterns, a portion of the tubing was initially filled with the first bioink and allowed to self-assemble for 30 min which was long enough to allow the collagen to gel and solidify but not sufficient for the cells to adhere to the ECM and initiate substantial shrinkage. Next, the second bioink was introduced into the rest of the tubing which then subsequently also self-assembled forming an axially patterned construct. The interface between the two cell regions in the tissue construct formed after shrinkage induced by the cell attachment to the ECM was found to be precise and capable of withstanding high internal tension. Delay in addition of second phase resulted in two separate unfused regions that due to high contractile force were positioned distant from each other (FIG. 21b). Thus, addition of second bioink in the axial configuration should be done right after completion of gelation process for the first bioink. If it is added earlier, a well-defined border between two regions may not be formed and if it is added long after this point, a firm junction may not be formed, and due to force exerted by shrinkage of the constructs they will tear apart. Concentric or radial patterning of cells was formed by initially filling the entire space between the pins with the first bioink followed by a longer incubation time (˜2 hrs) so that the construct shrank to nearly half of the final stable size. At this point the excess medium was extracted and replaced with the second bioink containing a different cell type and/or ECM combination (FIG. 21a) which then proceeded to self-assemble around the partially assembled first layer in an annular fashion. Formation of second layer around the first layer at the anchor pins is shown in FIG. 21c. In radial mode, by decreasing the cell number and increasing the CMR single layer cell coverage can be potentially made to cover the inner cell construct for example to mimic blood-brain barrier. These patterning can also be used to create constructs with different types of ECMs in different locations. While presence of collagen is necessary for formation of stable structures, other types of ECM can be mixed with it to provide more favorable environment for different cells in each region or for example to study migration of cells from one region with one type of ECM to the other. Such constructs allow direct contact between different cell types at the interface and paracrine interactions for the rest of the cells in different layers. The paracrine interactions can also be modeled in a unilateral direction by forming the constructs in two separate tubing and connecting them using interconnects. Applying a small fluid flow will allow exposure of the downstream construct to the paracrine signaling while preventing it in the upstream construct. Such cell patterning can also be useful in applications such as controlled release of pharmaceuticals.

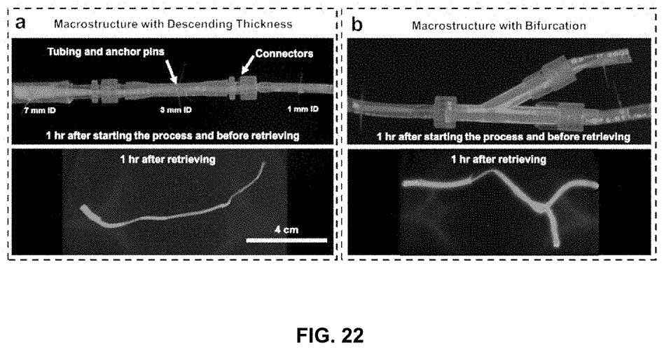

Spherical constructs have been widely used, for example in the case of spheroids, mostly due to ease of fabrication for applications such as modeling the initial avascular state of cancerous tissues, but this format can lead to formation of necrotic core that is not favorable for other applications including modeling physiological conditions of different tissues. An alternative and elegant tissue structure would be cylindrical or tubular structures that can be extended along their axial dimension to have a larger volume without increase in the radial direction in order to avoid formation of necrotic cores. Control over the radial dimensions of such structures affect the mass transport fluxes within the construct and could be used to create unique biochemical environments. Here such long tubular macrostructures with different thicknesses in different regions were fabricated (FIG. 22a) by connecting silicone tubing with different IDs, using appropriate connectors, inserting anchor pins in each of them and then filling the entire construct with the bioink. In under an hour, the cells and ECM rapidly assembled to form constructs that are several centimeters in length but have different diameters in the various sections. The constructs were robust enough that they can be retrieved from the tubing using tweezers and were strong enough to support their own weight. These types of constructs provide different mass transfer conditions in different sections and can be used as in vitro models or as cell delivery vehicles for in vivo implantation. Extrusion printing of bioinks containing hydrogels and cells can be used to form long tubular constructs but they typically have low cell density (less than a few million cells/mL). Newer extrusion techniques with lower speed can produce high density constructs with radial and axial patterning, but have difficulty in creating branching networks and require specialized equipment. Alternatively, the hanging drop method has been modified with patterned substrates in rectangularly designed hydrophilic regions to confine cells in a semi-cylindrical fashion in order to assemble ECM-free fibers. This interesting approach is an advancement over the traditional hanging drop method but is still limited in its ability to form multicellular patterns radially or axially. Furthermore, it requires specialized substrates and long assembly times. The approach described herein represents a simple yet robust method that can be adapted easily to produce macro tissues of almost any length from multiple cell types with the ability to create branching structures (FIG. 22b) very easily (single step) without the use of expensive equipment or complicated operations.

The branching structures shown here (FIG. 22b) are particularly important as complex interactions between different tissues can be simulated by fabricating each tissue separately in a tubing and then simply connecting them using appropriate Y-shaped connectors. These conformations allow more complex interactions where the paracrine signalling of two different tissues can be simultaneously exposed to a common down stream tissue or inversely the signaling from a common upstream tissue could provide exposure to several downstream tissues while they do not affect each other. By repeating these connections, a more complex fluidic network can be developed that can provide physiologically relevant paracrine interactions between multiple tissue types in a simple and robust way without the use of any complex microfabrication processes. By using different cell densities in each tubing in the branched network or connecting different number of tubing containing each cell type a more accurate model of interaction between different tissues and organs can be created using proper allosteric scaling. After retrieving from the tubing, a noticeable shrinkage may occur but the constructs preserve their premeditated morphology.

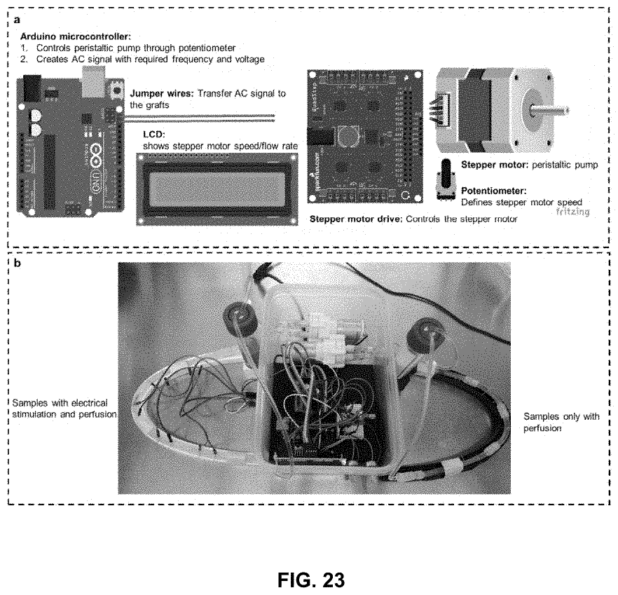

Dynamic Environment. In addition to 3D cell-cell and cell-ECM interactions and paracrine activities, biophysical signals such as mechanical or electrical stimulation play an important role in recreating the in vivo-like microenvironments that determine the functioning of tissues. The use of metal pins as anchors provided the ability to apply electrical stimulus to the tissue construct during different assembly and development phases. In addition, due to the self assembly and the contraction of the forming tissue construct that are constrained by the rigid pins, a time varying and auto-regulating mechanical stimuli is also applied on the construct. Similarly, the flexibility of the silicone tubing as well as the ability to perfuse the annular region between the tube and the tissue provided the ability to introduce active and dynamic mechanical stimulus and perfusion of fluids. A bioreactor (FIG. 23) was designed to apply electrical field (up to 5 V/cm with 50 Hz frequency) to the constructs through the anchor pins and to perfuse the growth medium to avoid waste accumulation and apply shear force. Using a microcontroller and additional pins in different locations, a range of different electrical signals can be applied at different locations and multiple assays can be conducted while continuity of the tissue construct and its exposure to nutrients and drugs are preserved.

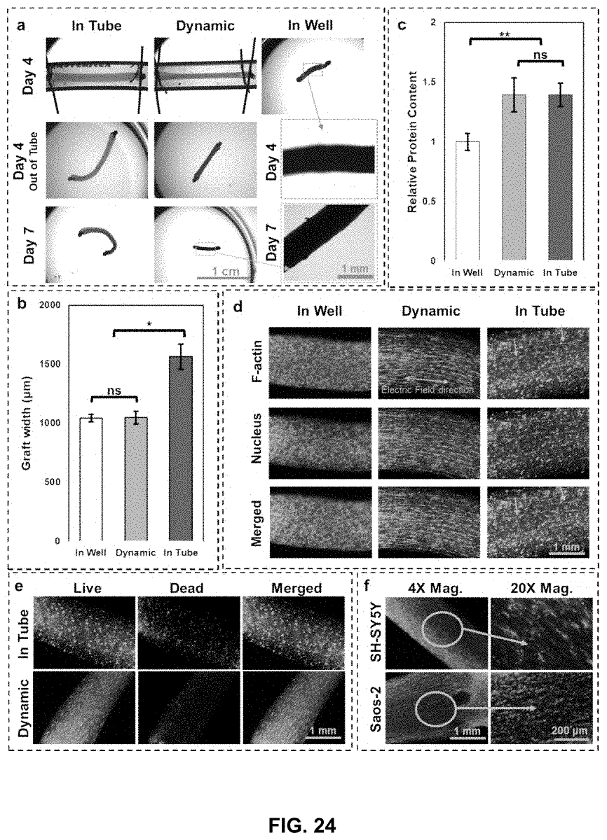

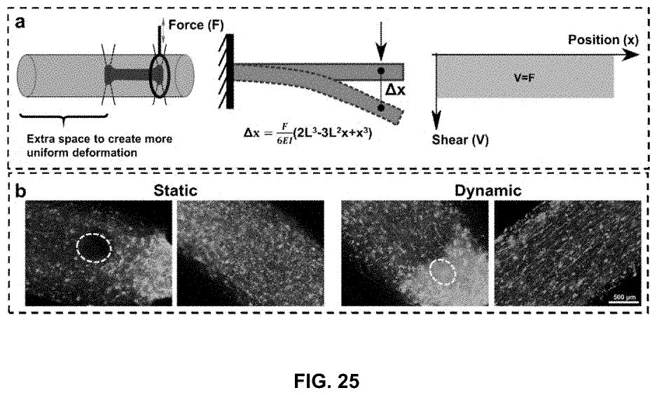

Importance of dynamic environment on cell function was studied by studying effect of electric field on differentiation and maturation of myoblast cells as well as their ECM deposition. For this purpose, muscle tissue constructs (formed using C2C12 cells, 1:3 CMR, 2×10⁶ cells/mL, and 2 cm apart pins) that were formed in their growth medium and subsequently their differentiation into mature skeletal muscle cells in the form of multinucleated myofibers, in three different conditions were compared. Cellular behavior in samples formed in the tubular constructs without subsequent confinement to the anchor pins was studied by transferring the formed tubular constructs to 6 well plates containing differentiation medium (“In Well” group), 24 hrs after the process started. Effect of being confined to anchor pins on cell behavior was studied by keeping the formed tissue samples in the tubing (“In Tube” group) and switching to differentiation medium. Effect of electrical stimulation on this process was studied by applying electric field to the anchored samples in the tubing while they were exposed to differentiation medium (“Dynamic” group). Grafts in these conditions were compared 3 days later (4 days in culture in total) (FIG. 24). Bright field images of samples at day 4 (FIG. 24a) showed that “In Tube” group samples had significantly higher thicknesses (1563±105 m) compared to “Dynamic” and “In Well” samples which were not significantly different from each other (1047±55 and 1042±31 m respectively) (FIG. 24b). Measurement of total protein content of the constructs using Pierce BCA assay showed that both “Dynamic” and “In Tube” samples were similar to each other in protein content (FIG. 24c) which was significantly higher as compared to “In Well” samples (˜1.4 times higher). At day 4 “In Tube” and “Dynamic” samples were retrieved from the tubing and were kept in 6 well plate in differentiation medium for 3 more days. Immediately after retrieval, “Dynamic” samples showed a detectable shrinkage while it was much lower for “In Tube” ones. After 3 more days in culture, more shrinkage was observed for “Dynamic” samples while “In Tube” ones showed a small amount of shrinkage. Higher magnification imaging during the first 3 days of differentiation showed that a high number of cells disaggregated from “In Well” group and proliferated on the well surface (FIG. 24a) while such disaggregation was not seen in the case of “In Tube” and “Dynamic” constructs during the 3 days of culture after being retrieved from tubing. Staining for F-actin in the constructs using phalloidin at day 4 (FIG. 24d), revealed that although samples in all three groups were treated with the same differentiation medium, cells in the “In Well” group did not fuse and did not form multinucleated fibers unlike samples in the “Dynamic” group that showed formation of fibers aligned in the direction of electric field (perpendicular to the anchor pins). Samples in the “In Tube” group, which were exposed to mechanical constriction, had a few fibers formed which were very short compared to the ones in the “Dynamic” group. Presence of electrical stimulation in “Dynamic” group did not affect the protein content and therefore ECM production in those samples as compared with the “In Tube” group where there was no electrical stimulation (FIG. 24c), but it did induce more extensive fiber formation and maturation of skeletal muscle cells. This shows that various stimulation that are important to obtain morphological features seen in natural tissues can be induced in this method easily. Presence of anchor pins provided a continuous mechanical strain to the developing construct which stabilized the construct and prevented cells from disaggregating. Live/dead staining of the “In Tube” and “Dynamic” samples right after retrieval from the tubing at day 4 showed fewer dead cells in the “Dynamic” condition (FIG. 24e) which shows not only the “Dynamic” environment promoted the differentiation and maturation of cells, it also preserved their viability as well. There are slightly more dead cells in the “In Tube” group. Although electrical stimulation and perfusion have been applied previously to skeletal muscle cells to study myofiber formation, this method allows simultaneous application of all three stimuli—perfusion, electrical and mechanical—along with control over tissue interfaces, environmental factors such as construct size and compactness, as well as a fast process with little to no effect on cell viability. Electrical stimulation while greatly affected the cell alignment and fiber formation, did not influence the protein content of samples. Constructs kept shrinking over time outside the constriction of tubing and its anchors, cells did not show fusion and some of the cells even escaped the fiber on to the culture plate. Samples exposed only to the anchor pins showed some fiber formation and did not show much shrinkage and cell escape after retrieving from the tubing. Samples in “Dynamic” environment showed full fiber formation, had more shrinkage after retrieving from the tubing and no cell break out was observed.

Other tissue constructs including neural (formed using SH-SY5Y neuroblastoma cells) and osseous (formed from Saos-2 osteosarcoma cells) also demonstrated cellular alignment with the electrical stimulus in our tissue formation method (FIG. 24f). These constructs were kept in culture for 8 days and were able to maintain their integrity despite observation of further shrinkage. Although the effect of electrical field on bone cells have been previously studied in 2D cultures, cells cultured on scaffolds, or substrates, here in situ cellular alignment of osteoblast-like cells in a truly 3D culture system composed only of cells and ECM is shown. Such alignment can potentially be used to mimic the anisotropic microstructure of bone that influences its behavior resulting in anisotropic viscoelastic properties. Similarly, alignment of neuronal cells along with electric field lines have been demonstrated previously in 2D culture systems or on scaffolds as well as in loosely packed hydrogel based constructs. However, this method demonstrates the ability to create highly dense and aligned neuronal tissue constructs without pre-fabricated scaffolds which can be used to form neural tube bundles for the use in regenerative medicine applications.

In addition to electrical stimulation, additional biophysical stimulation can also be applied in this system. For instance, other types of stimuli including the perfusion of medium that generates shear force of the fluid flow on the cells on the outer layer of the tubular construct and the mechanical bending of the tubing that translates to the mechanical deformation of the constructs including stretching or compression can be shown using the method described herein. Wave-like mechanical deformation can be created through induction of in the tissue graft by controlling the flow rate of the medium as well. Effect of this mechanical deformation on maturation of skeletal muscle cells was studied by forming the C2C12 constructs and creating the dynamic environment by applying mechanical stimulation by bending the tubing for 2 hr every day for 3 days. Mechanical deformation was started one day after grafts were formed and transferred to differentiation medium. The tubing and graft inside it can be treated as a beam that is fixed on one side and is deflected using a concentrated force on the other end. There is a uniform shear force applied to all cross-sections of the sample across the length of the graft and while there is a cubic relation between deformation and position. In order to create more uniform deformation in the graft, in FIG. 25, a 3 cm extra space between the left fixed side of the tubing and the graft was allowed. More fibers were observed in those constructs exposed to mechanical deformation (FIG. 25) as compared to those without stimulation. However, fewer fibers were formed under this mechanical stimulation compared with electrical stimulation (FIG. 24d), which could be because of the shorter (only 2 hr of mechanical stimulation was done every day) duration of stimulation compared with electrical one (applied continuously). Similar effect of mechanical deformation on cellular alignment and fiber formation of skeletal muscle cells and their maturation in 3D culture systems have been previously observed but independent of stimulation mode (chemical, mechanical, or electrical) or ECM type, it has been shown that once such mature skeletal muscle cell constructs that show dense and highly organized fibers are formed, the constructs can be actuated and will exert forces that can be used for applications such as soft biorobotics and bioactuators.

This technique is also compatible with high throughput screening applications. For example, a large number of constructs can be formed in the same tubing by inserting more than just two anchor pins or by connecting different construct containing tubing to each other in series. This could increase the nutrient consumption and by-product accumulation rate and adjustments to the flow rate of the medium or size of the tubing needs to be done to properly support cellular behavior. Alternatively, connections in parallel can be also used in case perfusion is not desired. This will isolate the metabolic impact of one tissue type on the other. A combination of series and parallel connections can be introduced to replicate the ratio of metabolic outputs of different tissue types in the body.

### CONCLUSION

A new and simple biofabrication technique for rapid formation of collagenous, tubular, macroscale tissue constructs has been developed. The method allows for formation of complex tubular shapes and branching networks while providing the flexibility to control positioning of different cell types in predefined patterns, at high densities and with clear interfaces that can mimic in vivo like environments. The fabrication process is low cost, simple, and easy to adapt to create various tissue geometries and allosteric scaling. It can also be used to apply various biophysical stimuli such as mechanical deformation, fluid shear, and electric field separately or in conjunction to create a dynamic environment as well. A variety of cell types including endothelial, epithelial, skeletal muscle cells, bone cells, and neuronal cells are amenable to this method, and multicellular structures can be created by radial or axial patterning. To demonstrate the efficacy of this method, aligned muscle, neural, and bone tissues were constructed. Macrostructures (several centimeters in length) with complex patterns such as the columns with different thicknesses in different regions and bifurcated constructs which can be used as cellular constructs or in vitro models were rapidly constructed. By providing both the biochemical and the biophysical environment and the ability to direct complex paracrine interactions between different segments using fluid flow, these systems can serve as a versatile tool for biomedical researchers understanding disease mechanisms and discovering new drugs.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Tables

TABLE 1
Code used for programming the microcontroller
that controls the bioreactor.

//define pins for the peristaltic pumps
int EnA=10; //yellow wire
int in1=9; //orange wire
int in2=8; //red wire
int EnB=11;
int in4=12;
int in3=13;
//output voltage for 1st pump
int potValue1=0 ;
long pwmOutput1=0;
int VoltOutput1=0;
int potValue2=0;
long pwmOutput2=0;
int VoltOutput2=0;
//using potentiometers to define speed of peristaltic pumps
int pot1=A1;
int p0t2=A2;
// a step wave between pins 6 and 7 (−5 to +5V) with a frequency
of 50Hz
int PinP = 6; //
int PinN = 7; //
int counter=0;
void setup( ) {
//controling 1st pump
pinMode(EnA, OUTPUT);
pinMode(in1, OUTPUT);
pinMode(in2, OUTPUT);
//controling 2nd pump
pinMode(EnB, OUTPUT);
pinMode(in3, OUTPUT);
pinMode(in4, OUTPUT);
pinMode(pot1, INPUT);
pinMode(pot2, INPUT);
//defining outputs for the Sin wave
pinMode(PinP1, OUTPUT);
pinMode(PinN1, OUTPUT);
pinMode(PinP2, OUTPUT);
pinMode(PinN2, OUTPUT);
}
void loop( ) {
//reading the potentiometer and defining the speed of 1st peristaltic
pump
potValue1 = analogRead(pot1); // Read potentiometer value
pwmOutput1 = map(potValue1, 0, 1023, 0 , 255); // Map the
potentiometer value from 0 to 255
analogWrite(EnA, pwmOutput1); // Send PWM signal to L298N
Enable pin
digitalWrite(in1, LOW);
digitalWrite(in2, HIGH);
potValue2 = analogRead(pot2); // Read potentiometer value
pwmOutput2 = map(potValue2, 0, 1023, 0 , 255); // Map the potentiometer value from 0 to 255
analogWrite(EnB, pwmOutput2); // Send PWM signal to L298N
Enable pin
digitalWrite(in3, LOW);
digitalWrite(in4, HIGH);
//creating AC signal
switcher( );
}
void switcher( ){
counter=counter+1;
if (counter%2==0){
digitalWrite(PinP, HIGH);
digitalWrite(PinN,LOW);
delay (10);
}else{
digitalWrite(PinN, HIGH);
digitalWrite(PinP,LOW);
delay (10);
}
}

### FULL CITATIONS FOR DOCUMENTS REFERRED TO IN THE APPLICATION

1. McGuigan, A. P., et al., Cell Encapsulation in Sub-mm Sized Gel Modules Using Replica Molding. PloS one, 2008. 3(7).
2. Legant, W. R., et al., Microfabricated tissue gauges to measure and manipulateforcesfrom 3D microtissues. Proc Natl Acad Sci USA, 2009. 106(25): p. 10097-102.
3. Sakar, M. S., et al., Cellular forces and matrix assembly coordinate fibrous tissue repair. Nature Communications, 2016. 7: p. 11036.
4. Turnbull, I. C., et al., Advancing functional engineered cardiac tissues toward a preclinical model ofhuman myocardium. FASEB J, 2014. 28(2): p. 644-54.
5. Osaki, T., S. G. M. Uzel, and R. D. Kamm, Microphysiological 3D model of amyotrophic lateral sclerosis (ALS) from human iPS-derived muscle cells and optogenetic motor neurons. Sci Adv, 2018. 4(10): p. eaat5847.

1. A method for preparing a construct comprising:
a) preparing a mixture of an extracellular matrix and a plurality of cells suspended in a first cell culture medium,
b) applying a crosslinking or gelation agent to the mixture,
c) depositing the mixture from b) into a mold of a defined shape,
d) allowing the extracellular matrix in the mixture in c) to crosslink or gel for a duration of about 1 hour to about 4 hours,
e) applying an additional cell culture medium to the mixture containing crosslinked or gelled extracellular matrix from d), and
f) allowing cell directed self-assembly of the mixture from e) for a duration of about 2 hours to about 10 hours to form a construct,
wherein the construct is a three-dimensional structure formed within the mold of the defined shape.

2. The method of claim 1, further comprising removing the construct from the mold.

3. The method of claim 1, wherein the construct retains the defined shape after removal from the mold.

4. The method of claim 1, wherein the extracellular matrix comprises a hydrogel, collagen, fibrin, laminin, elastin, alginate, gelatin, fibrinogen, chitosan, hyaluronan acid, polyethylene glycol, lactic acid, N-isopropyl acrylamide, glycoproteins, proteoglycans, basement membrane proteins, Matrigel™, Geltrex™, or combinations thereof.

5. The method of claim 1, wherein the ratio of the volume of the extracellular matrix to the volume of the first cell culture medium is between 1:1 and 1:10, optionally 1:1 to 1:6.

6. The method of claim 1, wherein the gelation agent is an alkaline substance.

7. The method of claim 1, wherein 10⁵ to 10¹⁰ cells/mL are suspended in the first cell culture medium.

8. The method of claim 1, wherein the extracellular matrix has a concentration of about 4 mg/mL to about 20 mg/mL.

9. The method of claim 1, wherein the plurality of cells comprise mammalian cells.

10. The method of claim 1, wherein the construct comprises about 10⁶ cells/mL to about 10¹⁰ cells/mL.

11. The method of claim 1, further comprising preparing at least one additional mixture of a second extracellular matrix and a second plurality of cells suspended in a second cell culture medium, wherein the second plurality of cells comprise at least one different cell type from the plurality of cells, applying a crosslinking or gelation agent to the additional mixture and depositing the additional mixture into the mold such that different cell types are spatially separated within the construct.

12. The method of claim 1, wherein the cell culture medium comprises a basal medium and optionally at least one supplement selected from the group consisting of plasma, serum, lymph, amniotic fluid, pleural fluid, growth factors, hormones, crude protein fractions, recombinant proteins, protein hydrolysates, synthetic polypeptide mixtures, tissue extracts and combinations thereof.

13. The method of claim 1, wherein the first cell culture medium and the additional cell culture medium are the same.

14. The method of claim 1, wherein the mold or parts of the mold comprise a material that is removed from the construct after the three-dimensional structure is formed, optionally wherein the material is extracted, dissolved or melted from the three-dimensional structure of the construct.

15. The method of claim 1, wherein the mold comprises a cell non-adhesive material, optionally polydimethylsiloxane.

16. The method of claim 1, wherein the mold defines the shape of a sphere, rod, tube, dumbbell, cuboid or combination thereof.

17. The method of claim 1, wherein the mold comprises a wire or rod that when removed from the construct after the three-dimensional structure is formed, results in a construct comprising a hollow interior space.

18. The method of claim 1, wherein at least one stimuli is applied to the mixture or the construct, optionally wherein the stimuli is a biophysical stimuli.

19. The method of claim 1, wherein the mold defines the shape of a tube and at least one elongated metal material is inserted into the mold.

20. The method of claim 19, wherein at least one stimuli is applied to the mixture or the construct via the at least one elongated metal material.

21. A construct prepared by the method of claim 1.
